(19) United States
(12) Patent Application Publication
(10) Pub. No.: US 2016/0262782 A1
(43) Pub. Date: Sep. 15, 2016

(54) SURGICAL INSTRUMENT FOR GRASPING AND CUTTING TISSUE

(71) Applicant: Take5 Endotech, Bunschoten-Spakenburg (NL)

(72) Inventors: MENNO KALMANN, Elspeet (NL); Wieger Kaptein, Bunschoten-Spakenburg (NL)

(73) Assignee: Take5 Endotech, Bunschoten-Spakenburg (NL)

(21) Appl. No.: 15/161,133

(22) Filed: May 20, 2016

Related U.S. Application Data
(63) Continuation-in-part of application No. 14/708,227, filed on May 9, 2015.
Application No. 14/708,227 is a continuation of application No. 13/935,836, filed on Jul. 5, 2013, now Pat. No. 9,066,744.
Application No. 13/935,836 is a continuation of application No. 13/751,071, filed on Jan. 26, 2013, now abandoned.
Application No. 13/751,071 is a division of application No. 12/322,729, filed on Feb. 6, 2009, now Pat. No. 8,398,673.
(60) Provisional application No. 62/180,633, filed on Jun. 17, 2015.
(60) Provisional application No. 61/066,063, filed on Feb. 15, 2008.

(51) Int. Cl. A61B 17/295; A61B 17/28; A61B 17/00

(52) U.S. Cl. CPC A61B 17/295; A61B 17/00234; A61B 17/282; A61B 17/2816; A61B 2017/00353; A61B 2017/00393; A61B 2017/2925

(57) ABSTRACT

A surgical instrument including a handle assembly, an elongated member, and first and second jaws positioned adjacent a distal portion of the elongated member. At least one of the first or second jaws is movable with respect to the other jaw. A first movable member is movable between first and second positions to effect jaw movement in a grasping action and a second movable member is movable between first and second positions to effect jaw movement in a cutting action. A selector is engageable with the handle assembly and movable from a first position to a second position, wherein in the first position the selector is in blocking engagement with the handle assembly to prevent jaw movement in the cutting action and in the second position the selector is in blocking engagement with the handle assembly to prevent jaw movement in the grasping action.

This application claims priority to provisional application Ser. No. 62/180,633, filed Jun. 17, 2015, and is a continuation in part of application Ser. No. 14/708,227, filed May 9, 2015, which is a continuation of application Ser. No. 13/935,836, filed Jul. 5, 2013, now U.S. Pat. No. 9,066,744, which is continuation of application Ser. No. 13/751,071, filed Jan. 26, 2013, now abandoned, which is a divisional of application Ser. No. 12/322,729, filed Feb. 6, 2009, now U.S. Pat. No. 8,398,673, which claims priority from provisional patent application Ser. No. 61/066,063, filed Feb. 15, 2008. The entire contents of each these applications are incorporated herein by reference.

### BACKGROUND OF THE INVENTION

1. Field of the Invention

This application relates to a surgical instrument and more particularly to a surgical instrument for both grasping and cutting tissue.

2. Background

In minimally invasive surgery, the surgical procedure is performed by access to the surgical site through one or more small incisions. The surgical site is visualized by an endoscope inserted through one of the incisions and various surgical instruments are inserted through the incisions to manipulate the tissue as desired. The advantages of minimally invasive surgery are well established which include improved cosmesis, reduced chance of infection, faster patient recovery time and lower hospital costs.

Minimally invasive surgical procedures typically require the tissue to be grasped and held or manipulated. These procedures also typically require the tissue to be severed. Currently, to perform cutting and grasping functions separate instrumentation is required. This results in the time consuming task of withdrawing one of the instruments from the incision (usually through an access port such as a trocar extending into the patient) and inserting another instrument through the port. Not only does this take up valuable surgical time, but there is an increased chance of infection by passage of the instruments outside the body. There is also the risk of damaging tissue as the second instrument is inserted and advanced to the surgical site.

In these minimally invasive procedures, oftentimes the grasper jaws are used to dissect tissue. This can be achieved by opening the jaws to dissect the tissue with their outer portion to create a working space for access to the surgical site. During this dissection and instrument advancement, a vessel may be encountered which requires severing to provide further access to the surgical site. The surgeon sometimes cauterizes the vessel to sever it, but such “excessive cauterization” could damage surrounding tissue. Another alternative utilized by the surgeon is to remove the grasper and insert a pair of scissors or shears to sever the vessel. This instrument exchange has the disadvantages enumerated above. Additionally, after severing, to continue dissection, the scissors would have to be removed and a grasper reinserted. If another vessel requires cutting, an exchange for a scissor would again be necessary. As can be appreciated, multiple instrument exchanges could occur, thereby multiplying the foregoing risks. Some surgeons might try to use the open scissor jaws for dissection, but there is a risk of inadvertent cutting of tissue, so exchanging for a grasper is preferred.

Another example where currently instrument exchange between a scissor and grasper is necessary is in laparoscopic cholecystectomy. In this procedure, typically open jaws of the grasper are used to dissect around the bile duct and then a clip applier extending through a different access opening applies one or more clips on each side of the target area of the duct. The surgeon then needs to remove the grasper and insert a scissor to cut between the clips. Next, the scissors need to be removed so that graspers can be inserted to remove the gall bladder. As can be appreciated, exchanges of the grasping and cutting instrument are required.

It would be advantageous to provide a single instrument which achieves both cutting and grasping which would thereby avoid the disadvantages enumerated above of instrument exchanges. This was recognized in U.S. Pat. Nos. 6,391,043 and 7,410,494, commonly owned with the present application. However, there are several disadvantages associated with the instruments of these two patents. The present invention advantageously overcomes the disadvantages of these instruments and provides an instrument for both cutting and grasping tissue which provides significant manufacturing and clinical advantages.

### SUMMARY OF THE INVENTION

The present invention provides an instrument capable of both cutting (severing) tissue and grasping tissue using the same pair of jaws. The instrument may also advantageously have a uniquely designed ergonomic handle assembly which eases manipulation of the instrument jaws. The instrument may also be designed to ensure the jaws are in their closed position when they are switched between the cutting and grasping functions. The instrument may also advantageously have a built in mechanism to prevent movement of the jaws in a cutting action when a grasping action is selected and prevent movement of the jaws in a grasping action when a cutting action is selected. The instrument may also be designed to apply a preload on the jaws to enhance their cutting action. The instrument may also be designed to be lightweight. One or more of the foregoing features can be provided in the various embodiments of the instrument.

The present invention provides in one aspect a surgical instrument for cutting and grasping tissue comprising a handle assembly disposed at a proximal portion of the instrument, an elongated member extending from the handle assembly, a first jaw positioned adjacent a distal portion of the elongated member, and a second jaw positioned adjacent the distal portion of the elongated member and mounted for movement with respect to the first jaw. A first movable member is operably associated with the second jaw and is movable between first and second positions to move the second jaw in a first direction about a first pivot axis in a grasping action. A second movable member is operably associated with the second jaw and movable between first and second positions to move the second jaw in a second direction different than the first direction and about a second pivot axis in a cutting action. A switch is positioned at the proximal portion of the instrument and a selecting member is actuated by movement of the switch. The selecting member is movable between a first position to enable movement of the second jaw in the first direction and prohibit movement in the second direction and a second position to enable movement of the second jaw in the second direction and prohibit movement in the first direction.

The instrument in some embodiments further comprises first and second locking mechanisms and the selecting member can comprise a rotatable linking member movable for selective engagement of one of the first and second locking mechanisms.

In some embodiments the instrument includes a first gear operably associated with the first movable member, a second gear operably associated with the second movable member, a first locking member engageable with the first gear and a second locking member engageable with the second gear, the selecting member selectively moving one of the first and second locking members into engagement with its respective gear.

In some embodiments, the first jaw has a first upper surface with a first edge and a second edge and the second jaw has a second lower surface with a third edge and a fourth edge, wherein in a cutting action, the first and third edges pass each other in substantially parallel planes and in a grasping action the first and third edges pivot away from each other in transverse planes. In some embodiments, the first pivot axis and the second pivot axes intersect and are substantially perpendicular.

The instrument may include a linkage mechanism having multiple links linking the switch to the selecting member.

In some embodiments, the instrument may include a safety mechanism operably associated with the switch to prevent movement of the switch if the jaws are not in a closed position.

In some embodiments, both the first jaw and second jaw are movable by the movable members.

In some embodiments, a first handle is operably associated with the first movable member, a second handle is operably associated with the second movable member, and the first movable member comprises a first tubular member and the second movable member comprises a second tubular member, the tubular members preferably being coaxial.

In accordance with another aspect, the present invention provides a surgical instrument for cutting and grasping tissue comprising an elongated member, a first jaw positioned adjacent a distal portion of the elongated member, and a second jaw positioned adjacent the distal portion of the elongated member and mounted for movement with respect to the first jaw. A first movable member is operably associated with the second jaw and is movable between first and second positions to move the second jaw in a first direction in a grasping action. A second movable member is operably associated with the second jaw and is movable between first and second positions to move the second jaw in a second direction different than the first direction in a cutting action. A switch is positioned at a proximal portion of the instrument. A handle assembly includes a stationary grip, a first actuator and a second actuator, wherein the first actuator is operably associated with the first movable member to move the second jaw in a grasping action and the second actuator is operably associated with the second movable member to move the second jaw in the cutting action.

In one embodiment, the first actuator is positioned proximal of the stationary grip and the second actuator is positioned distal of the stationary grip.

In some embodiments, the first actuator is operably associated with a first gear mechanism and the second actuator is operably associated with a second gear mechanism.

In some embodiments, the switch is movable between first and second positions, wherein in the first position the switch enables movement of the second jaw in the first direction and prohibits movement in the second direction and in the second position the switch enables movement of the second jaw in the second direction and prohibits movement in the first direction. In some embodiments, first and second locking elements are operably associated with the switch to prohibit movement of the first or second actuator, depending on the position of the switch.

In accordance with another aspect, the present invention provides a surgical instrument for performing first and second different functions on tissue comprising an elongated member, a first jaw positioned adjacent a distal portion of the elongated member, and a second jaw positioned adjacent the distal portion of the elongated member and mounted for movement with respect to the first jaw. The first and second jaws are movable between open and closed positions in a first orientation and are further movable between open and closed positions in a second different orientation. A first actuating mechanism moves the jaws in the first orientation and a second actuating mechanism moves the jaws in the second orientation. A switch is provided for choosing the first or second actuating mechanism, wherein the switch can be activated only when the jaws are in the closed position.

In one embodiment, the first function is cutting and the second function is grasping. In this embodiment, preferably the first jaw has a first upper surface with a first edge and a second edge and the second jaw has a second lower surface with a third edge and a fourth edge, wherein when the jaws move between open and closed positions in the first orientation to perform the cutting function, the first and third edges pass each other in substantially parallel planes and when the jaws move between open and closed positions in the second orientation in the grasping function, the first and third edges pivot away from each other in transverse planes.

In some embodiments, movement of the switch moves a link in a clockwise or counterclockwise direction to effect operative engagement of one of the actuating mechanisms to prevent movement thereof.

The instrument may in some embodiments include a safety member operably associated with the switch, the safety member movable between a first position to lock the switch against movement and a second position to allow movement of the switch, the safety automatically locking the switch when the jaws are in the open position. In one embodiment, the safety includes a sliding member engageable with the switch in a proximal position.

In accordance with another aspect, the present invention provides a surgical instrument for performing first and second different functions comprising a handle assembly disposed at a proximal portion of the instrument, an elongated member extending from the handle assembly, a first jaw positioned adjacent a distal portion of the elongated member and a second jaw positioned adjacent the distal portion of the elongated member and mounted for movement with respect to the first jaw. A first movable member is operably associated with the second jaw and movable between first and second positions to move the second jaw in a first direction about a first pivot axis to perform a first function on tissue. A second movable member is operably associated with the second jaw, the second movable member movable between first and second positions to move the second jaw in a second direction different than the first direction and about a second pivot axis to perform a second different function on tissue. The second movable member is positioned within the first movable member. A switch is positioned at the proximal portion of the instrument to switch the instrument between the first and second functions.

In one embodiment, the first jaw has a first projecting member extending in a first direction engageable by the first movable member and a second projecting member extending in a second different direction engageable by the second movable member and the second jaw has a third projecting member extending in a first direction engageable by the first movable member and a fourth projecting member extending in a second different direction engageable by the second movable member. In one embodiment, the first and third projecting members lie substantially along the same axis and the second and fourth projecting members lie substantially along the same axis.

The present invention also provides in another aspect a handle assembly for a surgical instrument having first and second jaws. The handle assembly comprises a stationary handle, a first actuator, a second actuator and a switch. The first actuator has a first movable finger loop configured to receive a thumb of the user and is operably associated with a first movable member to effect movement of at least one of the instrument jaws in a grasping action. The second actuator has a second movable finger loop and is operably associated with a second movable member to effect movement of at least one of the instrument jaws in a cutting action. The first and second actuators and the switch are all operable by a single hand of a user, wherein the switch cannot be activated by the single hand of the user unless the user releases the thumb engagement of the first finger loop.

The handle assembly may further comprise a stationary handle positioned between the first and second actuators having a third stationary finger loop.

The present invention also provides in accordance with another aspect a surgical method of grasping and cutting tissue with a single instrument comprising:

providing an instrument having first and second jaws movable in a first orientation to perform a grasping function and movable in a second orientation to perform a cutting function;

moving a first actuator of the instrument to move the jaws in the first orientation between closed and open positions to grasp tissue;

moving a second actuator of the instrument to move the jaws in a second orientation between closed and open positions to cut tissue; and

moving a switching mechanism between first and second positions to select the grasping or cutting function, wherein the switching mechanism can only be moved when the jaws are in the closed position.

In one embodiment, the step of moving the switching mechanism in the second position moves a first locking member into engagement with an advancing mechanism for moving the jaws in the first orientation. In one embodiment, the step of moving the switching mechanism in the first position moves a second locking member into engagement with an advancing mechanism for moving the jaws in the second orientation.

In accordance with another aspect of the present invention, a surgical instrument is provided comprising a handle assembly disposed at a proximal portion of the instrument, an elongated member extending from the handle assembly, a first jaw positioned adjacent a distal portion of the elongated member and a second jaw positioned adjacent the distal portion of the elongated member. At least one of the first or second jaws is movable with respect to the other jaw. A first movable member is operably associated with at least one of the first and second jaws and movable between first and second positions to effect jaw movement in a grasping action. A second movable member is operably associated with at least one of the first and second jaws and movable between first and second positions to effect jaw movement in a cutting action. A selector is engageable with the handle assembly and movable from a first position to a second position, wherein in the first position the selector is in blocking engagement with the handle assembly to prevent jaw movement in the cutting action and in the second position the selector is in blocking engagement with the handle assembly to prevent jaw movement in the grasping action.

In some embodiments, movement of at least one of the first and second jaws in the grasping action is about a first axis and movement of at least one of the first and second jaws in the cutting action is about a second different axis. In some embodiments, the first axis and second axis intersect and are substantially perpendicular.

In some embodiments, the selector is not movable from the first position to the second position if the jaws are in an open position.

The instrument may further include a first handle operably connected to the first movable member and movable in a first direction to effect jaw movement to an open position and movable in a second direction to effect jaw movement to a closed position in the grasping action. In some embodiments, the first handle is movable further in the second direction to apply increased tension to the first and second jaws prior to the cutting action which causes compression of the first and second jaws.

In some embodiments, the selector cannot be moved to the second position unless the first handle is moved to a position to apply increased tension to the jaws. The handle assembly can include an engagement surface engageable with the selector, the surface preventing movement of the selector. The selector can include a spring biasing the selector in the first position and the second position and retaining the selector in first and second positions.

In some embodiments, the first movable member includes a first tubular member and the second movable member includes a second tubular member, the first and second tubular members being coaxially positioned.

In accordance with another aspect of the present invention, a surgical instrument is provided for cutting and grasping tissue comprising an elongated member having a distal portion, a first jaw positioned adjacent the distal portion of the elongated member, and a second jaw positioned adjacent the distal portion of the elongated member and movable with respect to the first jaw. A first movable member is operably associated with at least the second jaw, the first movable member having a first position wherein the second jaw is spaced from the first jaw, a second position wherein the second jaw is moved toward to the first jaw in a grasping action, and a third position wherein the first and second jaws are brought under increased tension and bend. A second movable member is operably associated with at least the second jaw, the second movable member movable from a first position to a second position to move the second jaw toward the first jaw in a cutting action. A selector is positioned at a proximal portion of the instrument to enable movement of either the first movable member or the second movable member.

In some embodiments, the second movable member is movable only after the first movable member is in the third position. The first movable member can in some embodiments be a first handle operatively connected to a first tube movable axially to effect movement of at least the second jaw in the grasping action. The second movable member can in some embodiments can be a second handle operatively connected to a second tube movable axially to effect movement of at least the second jaw in the cutting action. In some embodiments, the first and second movable members are axially, slidable tubular members.

In some embodiments, the selector is movable to enable movement of the second movable member in a cutting action only after the first movable member has been moved to the third position.

In accordance with another aspect of the present invention, a surgical instrument actuation mechanism formed from one or more metal sheets is provided, the one or more metal sheets formed to have a first handle operable to effect a jaw grasping action, a second handle operable to effect a jaw cutting action, a first link operably connected to the first handle, a second link operably connected to the second handle, and a selector operably connected to the first handle and the second handle to select handle movement in the grasping action or the cutting action.

In some embodiments, the first handle formed from the one or more metal sheets includes first and second handles each having a finger loop and the second cutter handle formed from the one or more metal sheets includes first and second handles each having a finger loop. First and second side plates can also be formed from the one or more metal sheets.

### DETAILED DESCRIPTION OF THE DRAWINGS

Preferred embodiment(s) of the present disclosure are described herein with reference to the drawings wherein:

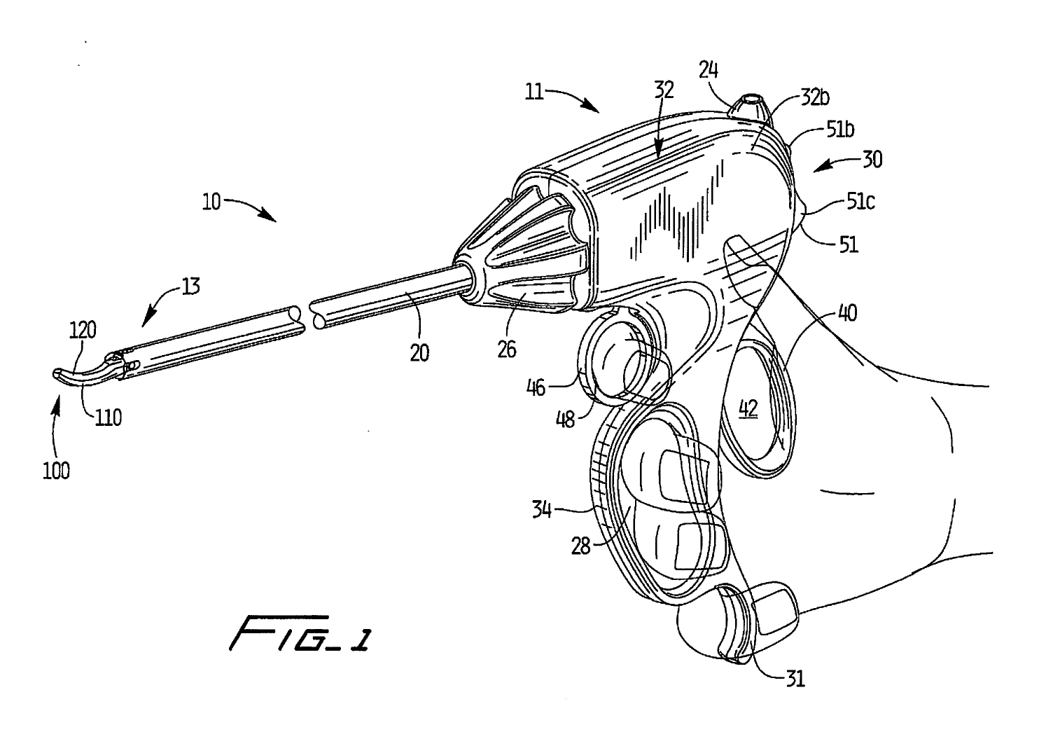

FIG. 1 is a perspective view of a first embodiment of the surgical instrument of the present invention showing the grasper handle and the cutter (scissor) handle in the at rest position with the jaws in the closed position;

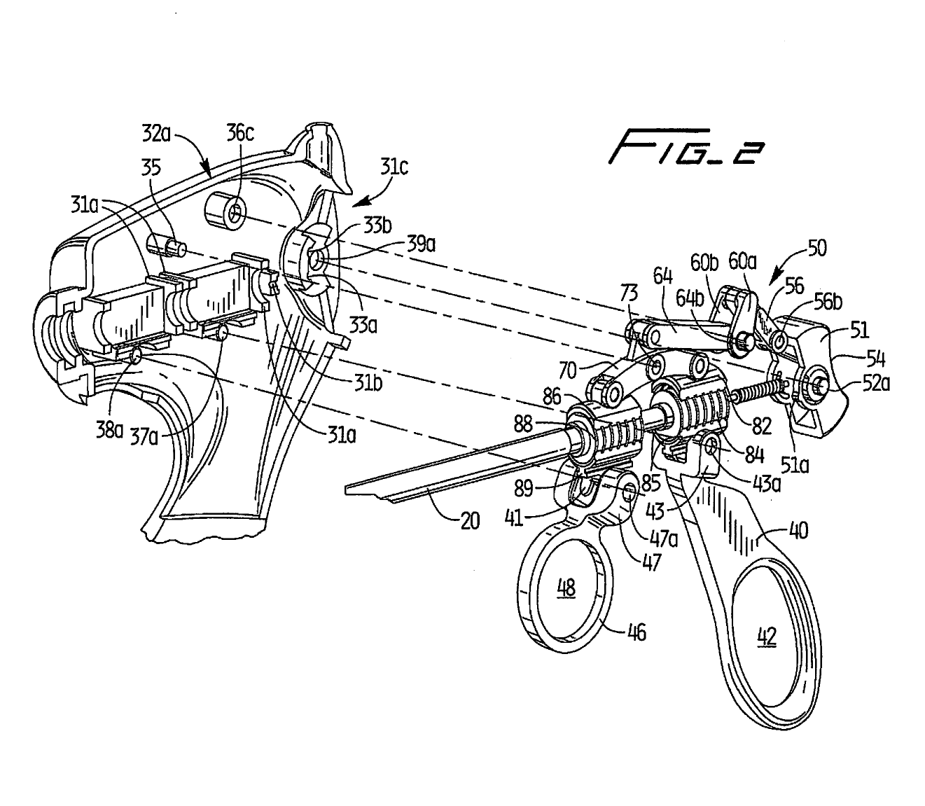

FIG. 2 is an exploded view of the handle assembly of the instrument of FIG. 1, showing one of the housing halves;

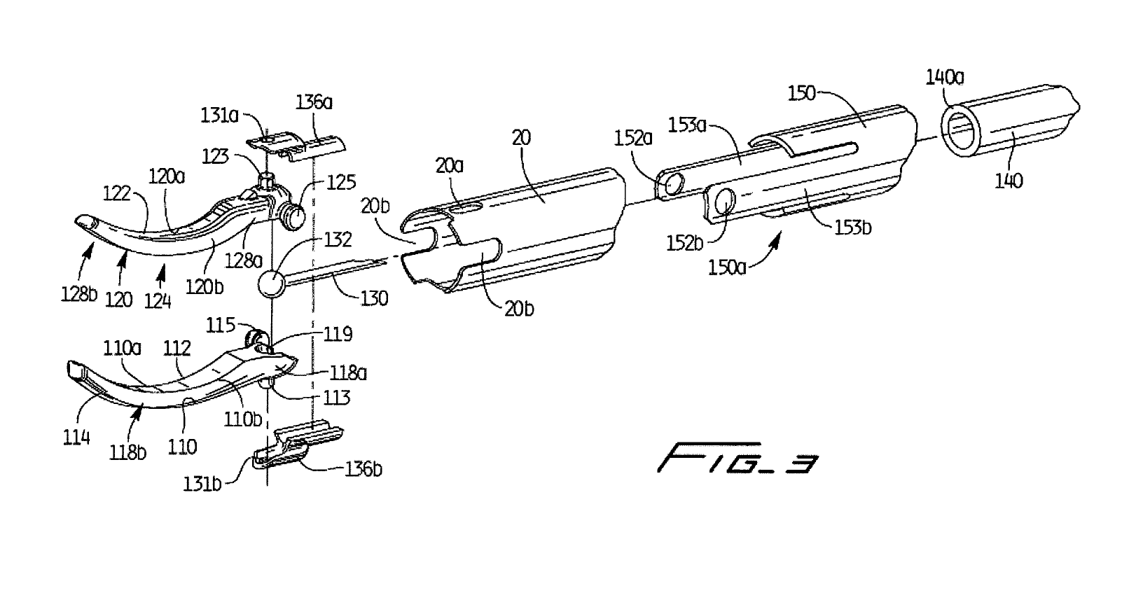

FIG. 3 is an exploded view of the jaw assembly and distal portions of the jaw actuators of the instrument of FIG. 1;

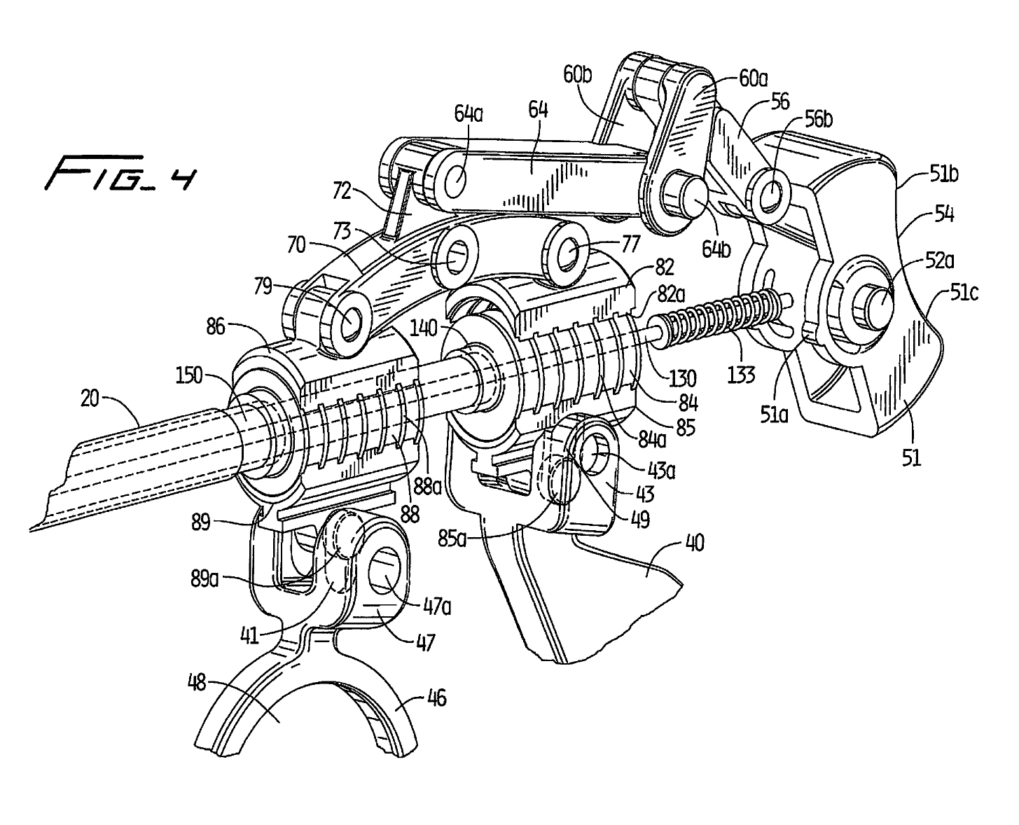

FIG. 4 is a perspective view of the actuators and switching mechanism corresponding to the position of the instrument of FIG. 1 and showing the switch in the grasping function position;

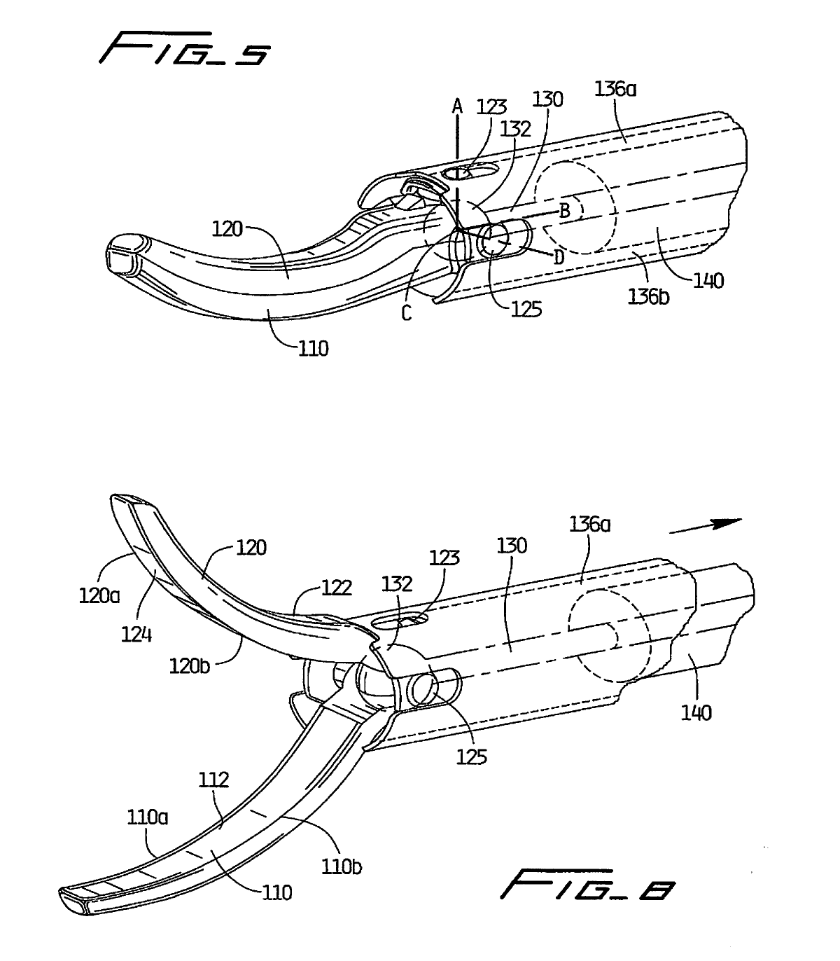

FIG. 5 is a perspective view of the jaws in the closed position corresponding to the position of the actuators of FIG. 4;

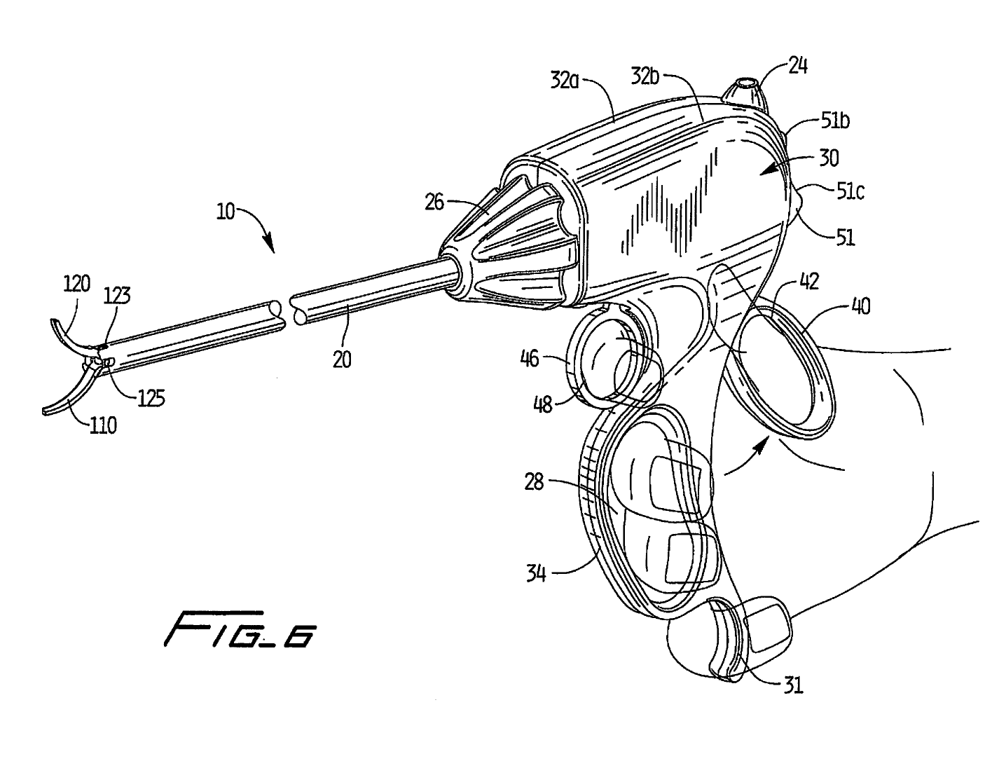

FIG. 6 is a perspective view similar to FIG. 1 showing the grasper handle in the retracted position and the jaws in an open grasping position, the switch remaining in the grasping function position;

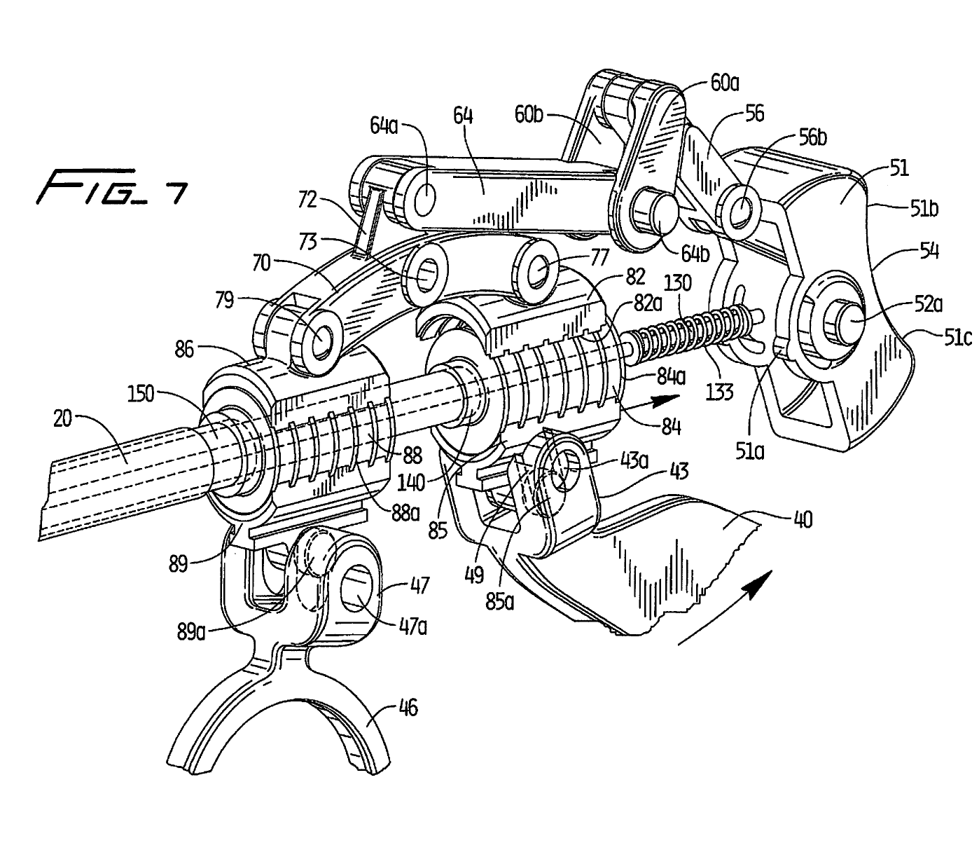

FIG. 7 is a view similar to FIG. 4 showing retraction of the grasper actuator to move the jaws to an open grasping position, and corresponding to the position of the instrument of FIG. 6;

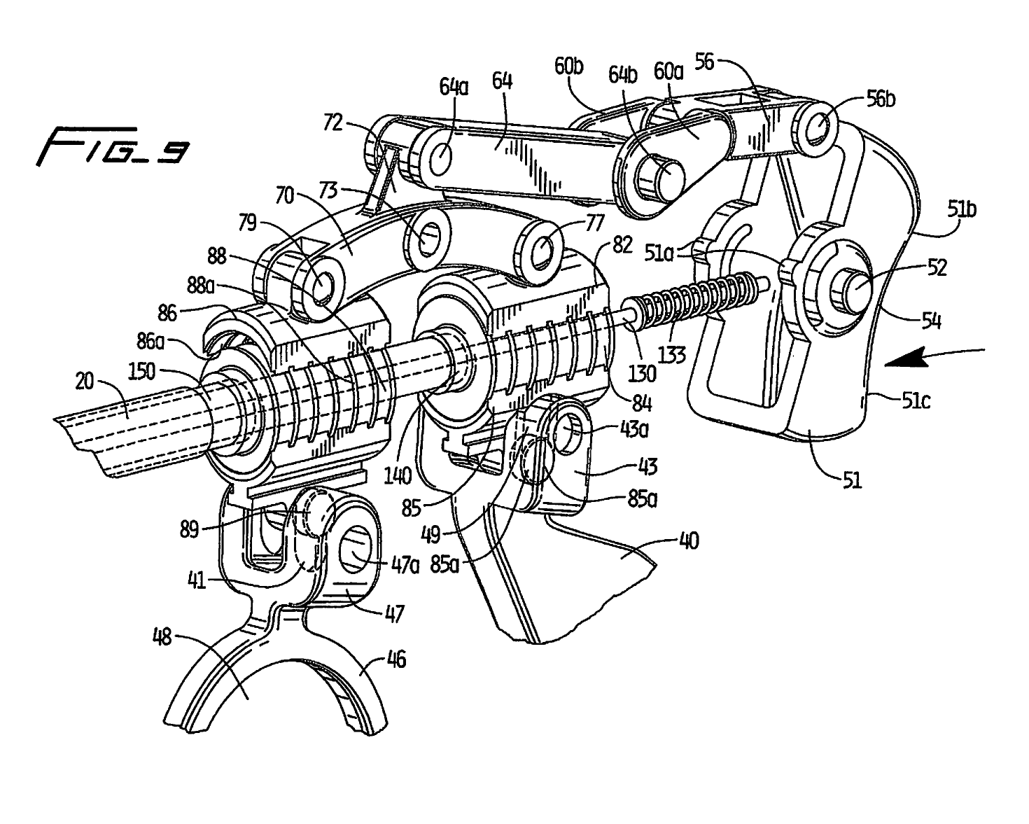

FIG. 8 is a perspective view of the jaws in the grasper open position corresponding to the actuator position of FIGS. 6 and 7;

FIG. 9 is a perspective view similar to FIG. 4 showing the switching mechanism in the cutting function position and the jaws in the closed position, and further showing the grasper spur gear locking the grasper gear to prevent movement of the grasper handle;

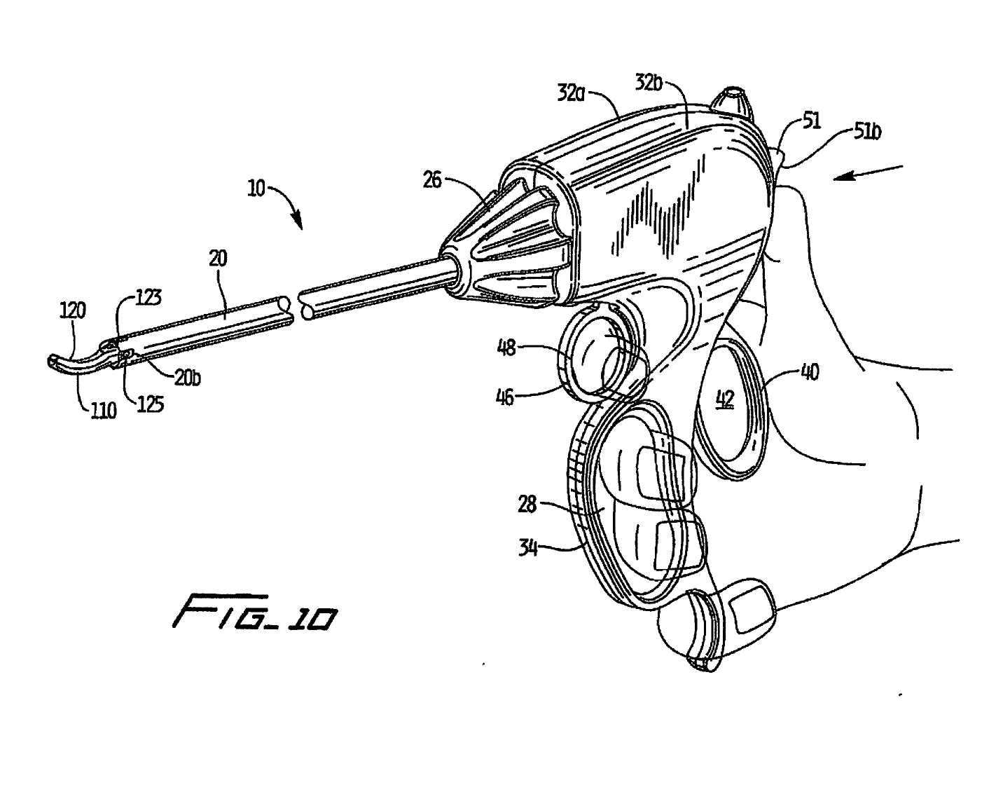

FIG. 10 is a perspective view of the instrument similar to FIG. 6 showing the grasper handle in the at rest position to close the jaws and the switching mechanism moved to the cutting function position;

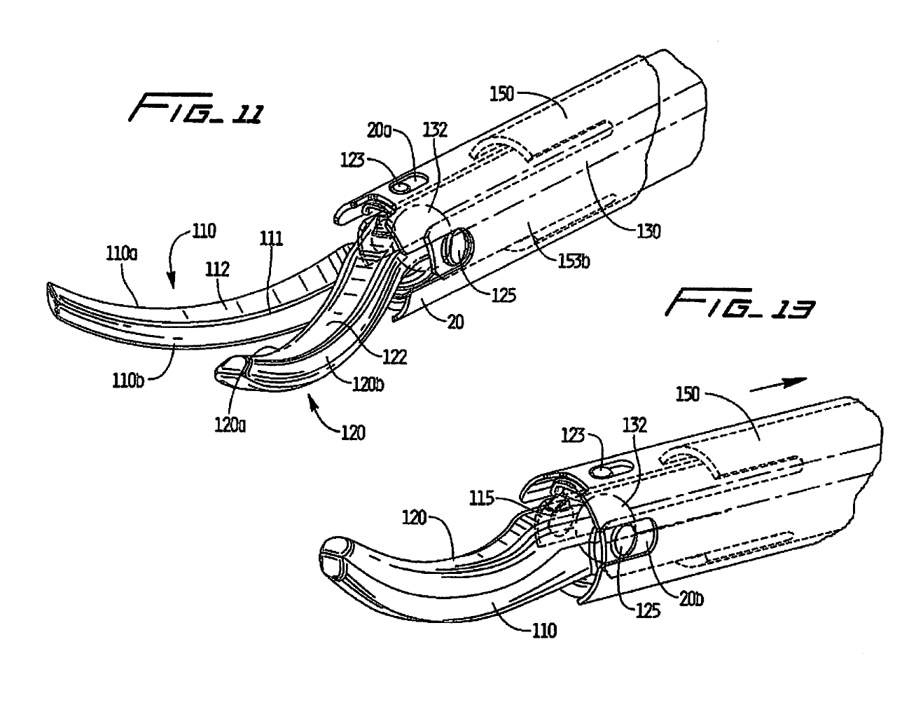

FIG. 11 is a perspective view of the jaws of the instrument of FIG. 1 in the open cutting position;

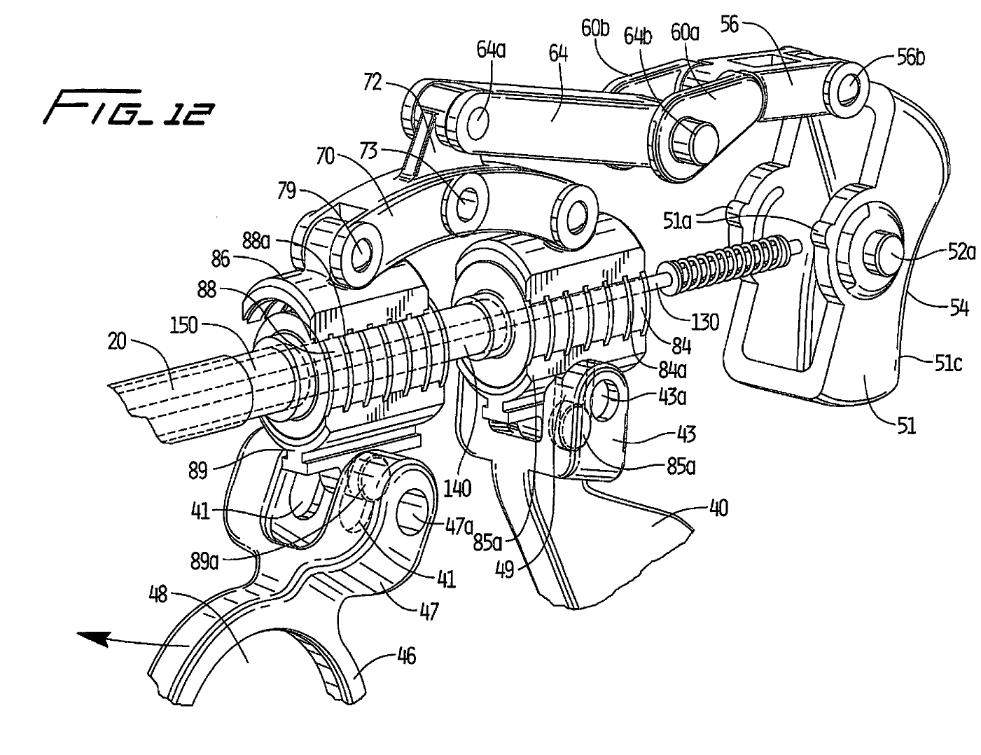

FIG. 12 is a view similar to FIG. 9 illustrating movement of the cutter handle to move the jaws to the closed position (the switch remaining in the cutting function position);

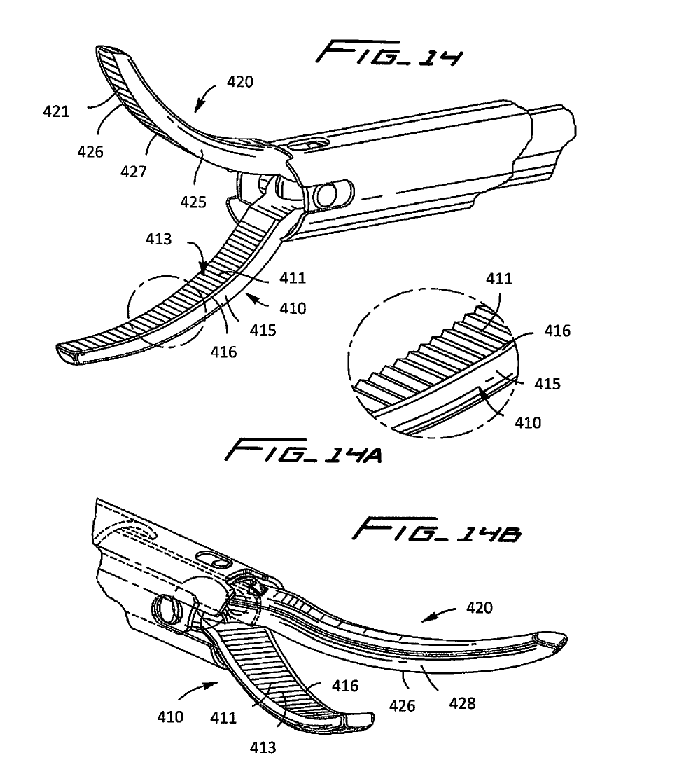

FIG. 13 is a perspective view of the jaws in the closed position after movement from the open position of FIG. 11 in a scissors action;

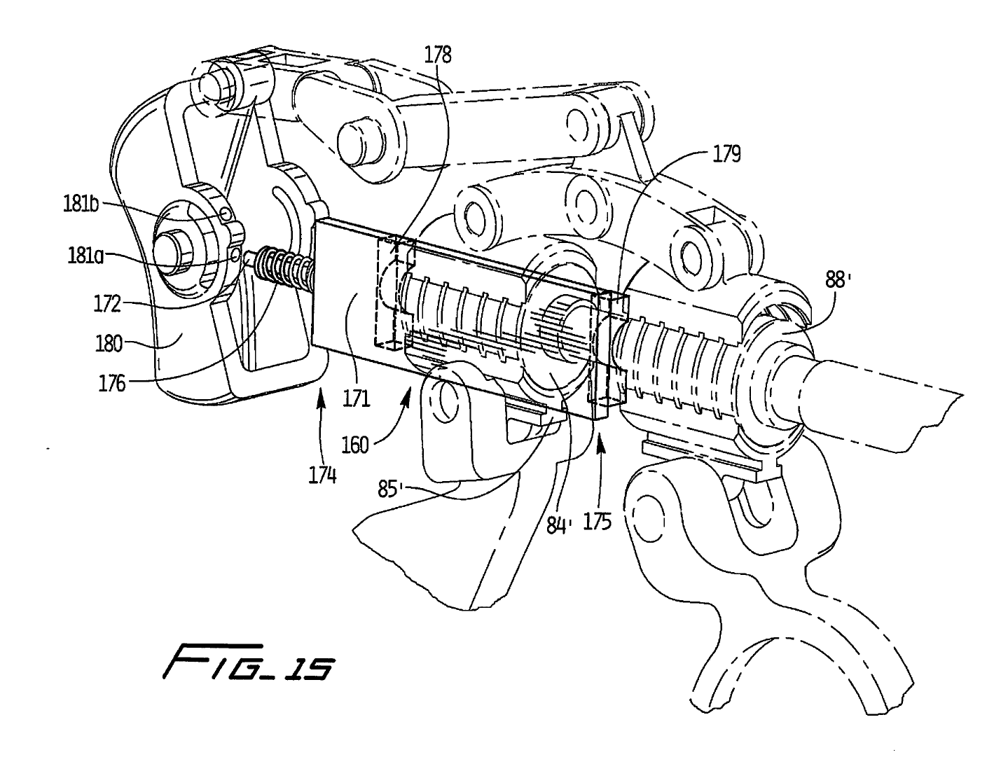

FIG. 14 is a perspective view of an alternate embodiment of the jaws having a series of teeth and a raised cutting surface;

FIG. 14A is a close up view of the area of detail of FIG. 14;

FIG. 14B is a perspective view similar to FIG. 14 showing the other side of the jaw assembly;

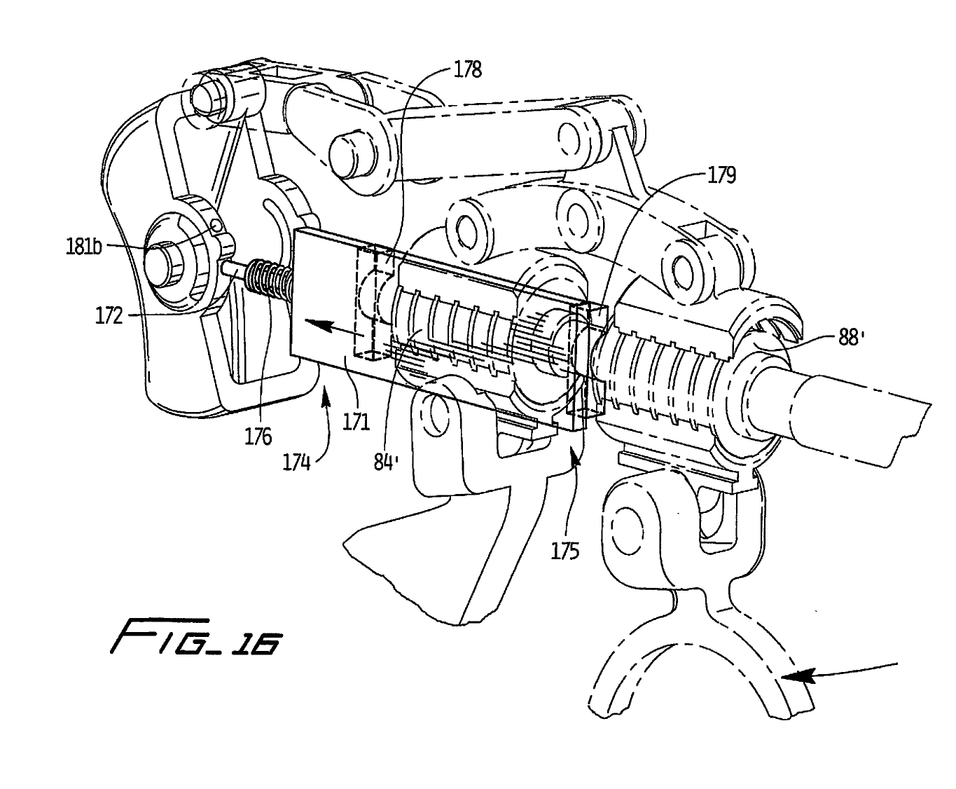

FIG. 15 is a perspective view of an alternate embodiment of the instrument of the present invention having a locking plate to prevent movement of the switching mechanism if the jaws are not in the closed position, the locking plate shown in the nonengaged position to allow movement of the switch;

FIG. 16 is a perspective view similar to FIG. 15 showing the locking plate in the engaged position to prevent movement of the switch from the cutting function position to the grasping function position;

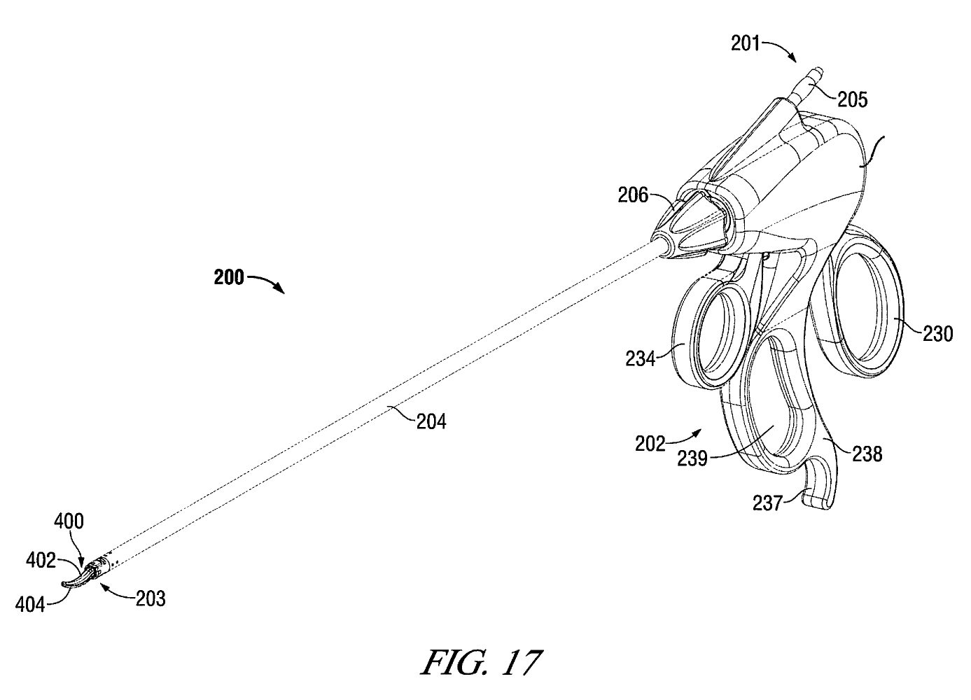

FIG. 17 is a perspective view of an alternate embodiment of the surgical instrument of the present invention showing the grasper handle and the cutter (scissor) handle in the at rest position with the jaws in the closed position;

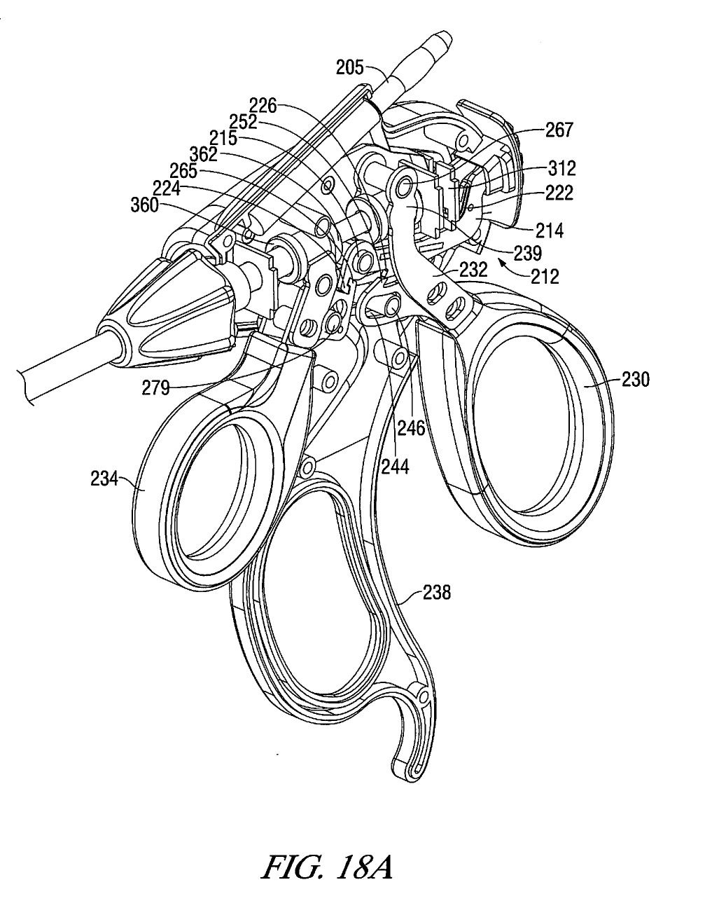

FIG. 18A is a perspective view of the handle portion of the instrument of FIG. 17, with one of the housing halves removed to show internal components, and the selector shown in the grasping function position;

FIG. 18B is a perspective view similar to FIG. 18A showing the selector in the cutting function position;

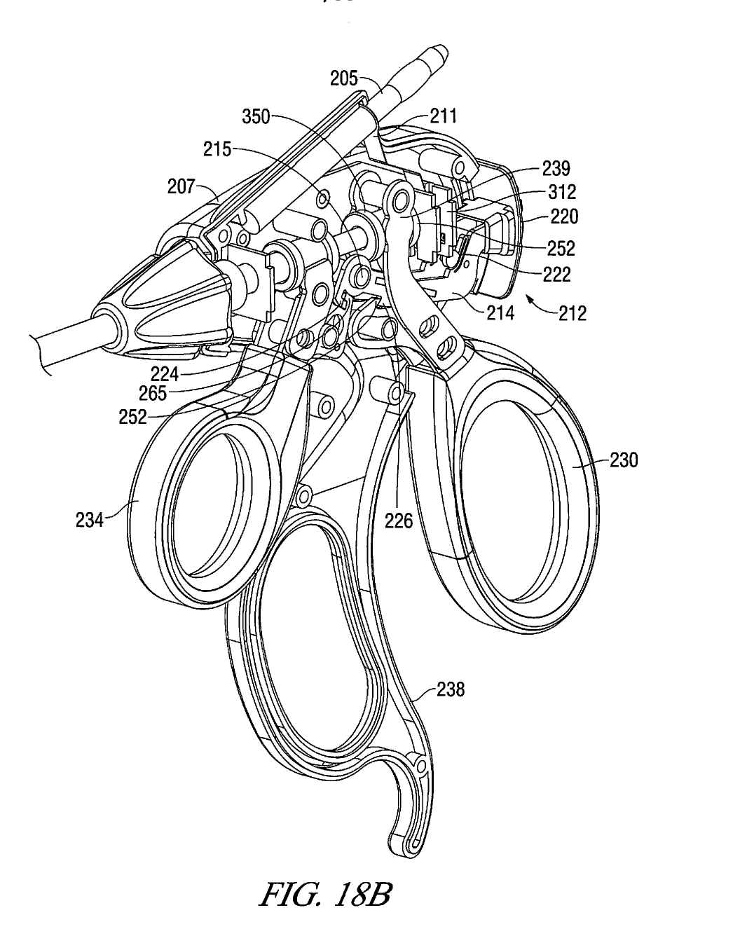

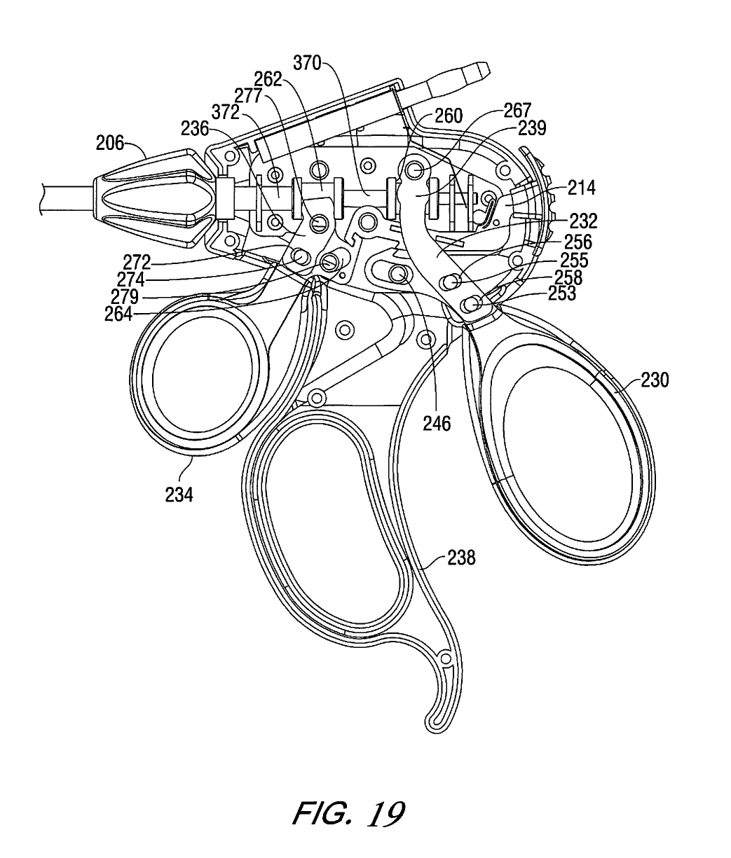

FIG. 19 is a side view of the handle portion of FIG. 18A in the grasping function position;

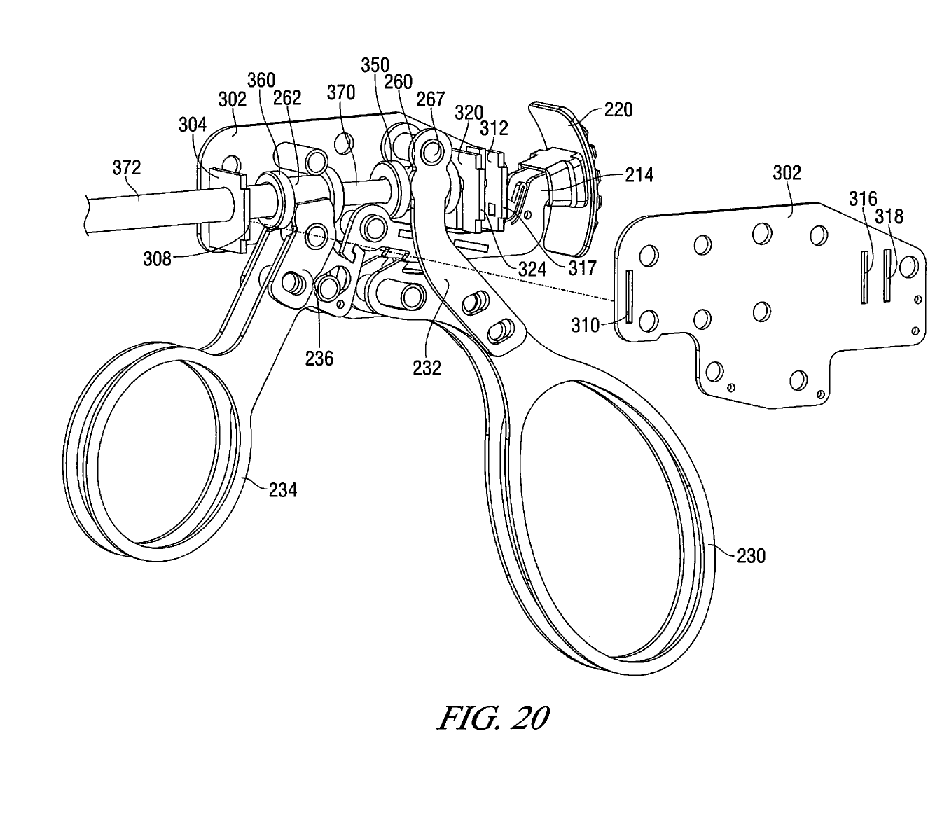

FIG. 20 is a perspective view of the handle portion in the position of FIG. 18A with certain components removed and one of the side plates shown separated from the handle assembly to show internal components;

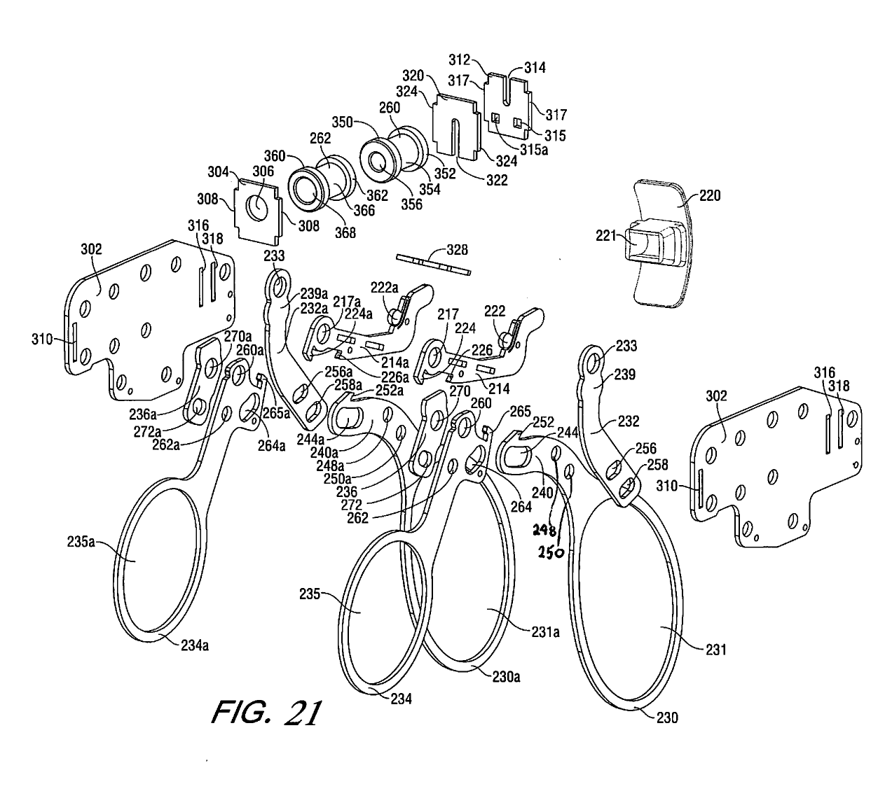

FIG. 21 is an exploded view of components of the handle portion of FIG. 18A;

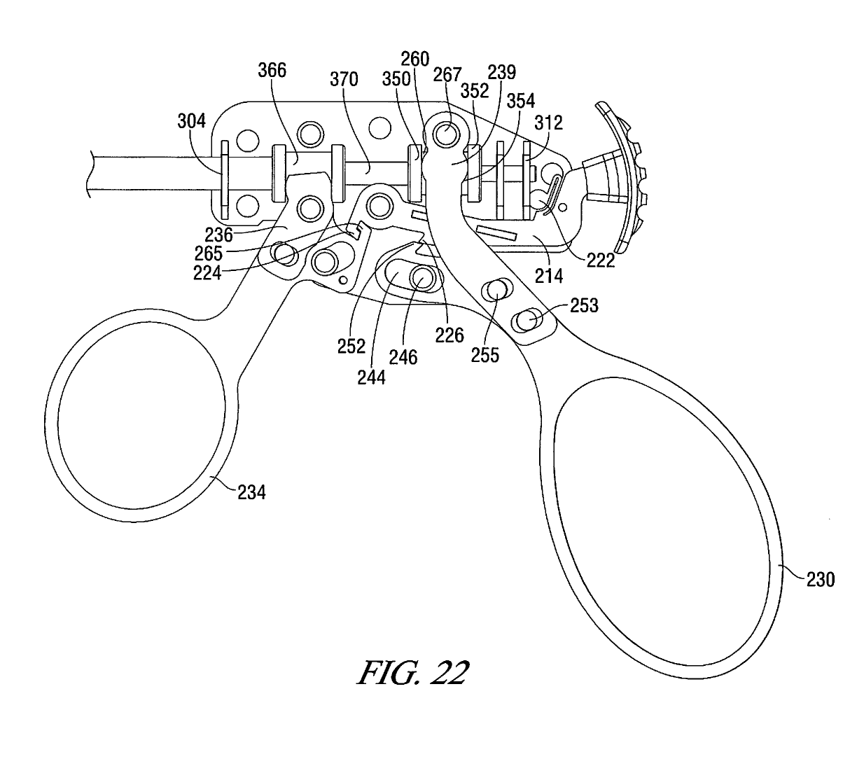

FIG. 22 is a side view of the handle portion with certain components removed for clarity, and showing the selector and handle in the grasping function position of FIG. 20;

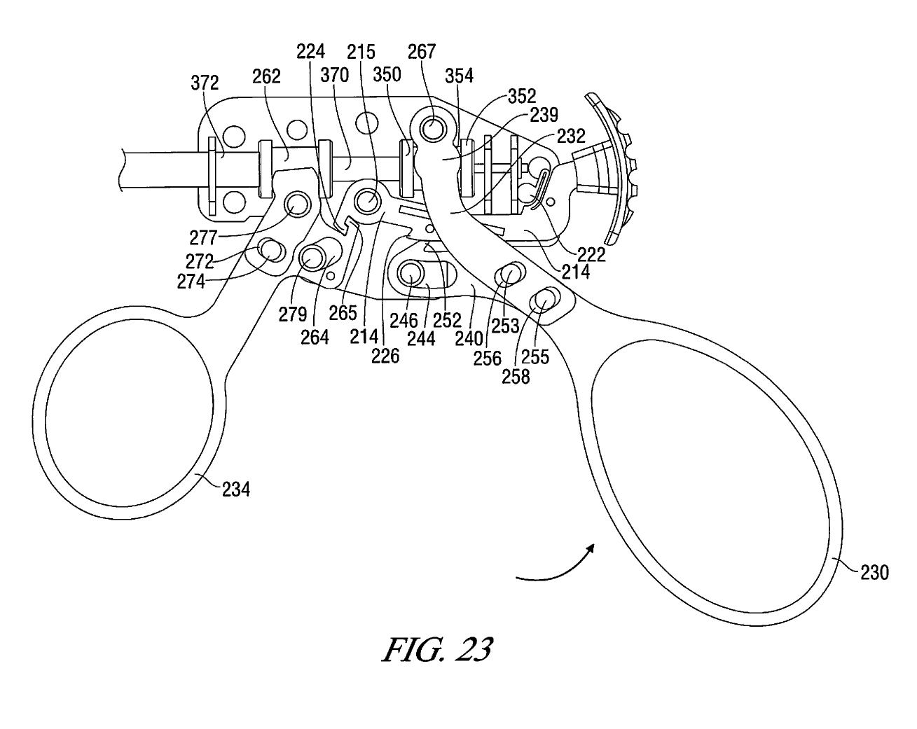

FIG. 23 is a side view similar to FIG. 22 showing the grasper handle moved proximally to retract the grasper tube to open the jaws in a grasping function;

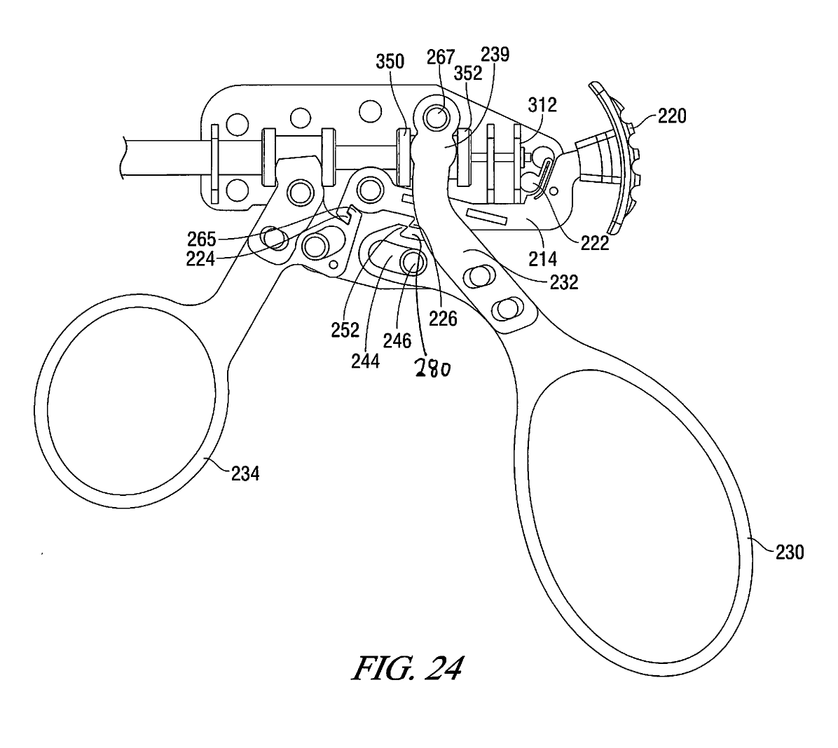

FIG. 24 is a side view similar to FIG. 22 showing movement of the grasper handle to a distal position to tension the jaws and enable movement of the selector to the cutting function position;

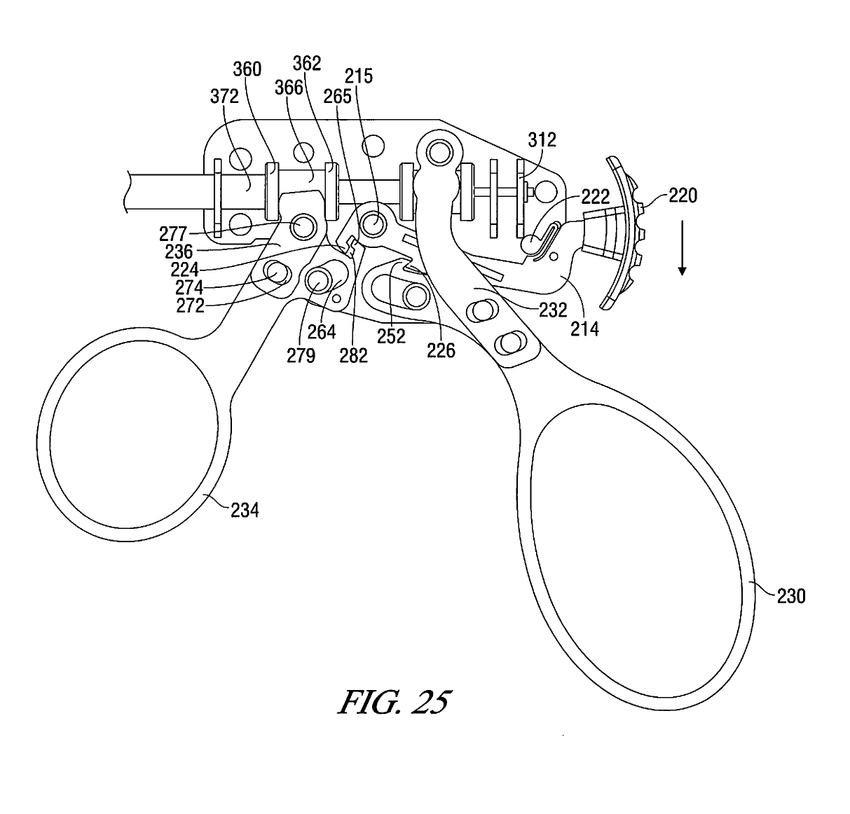

FIG. 25 is a side view similar to FIG. 24 showing movement of the selector to the cutting function position;

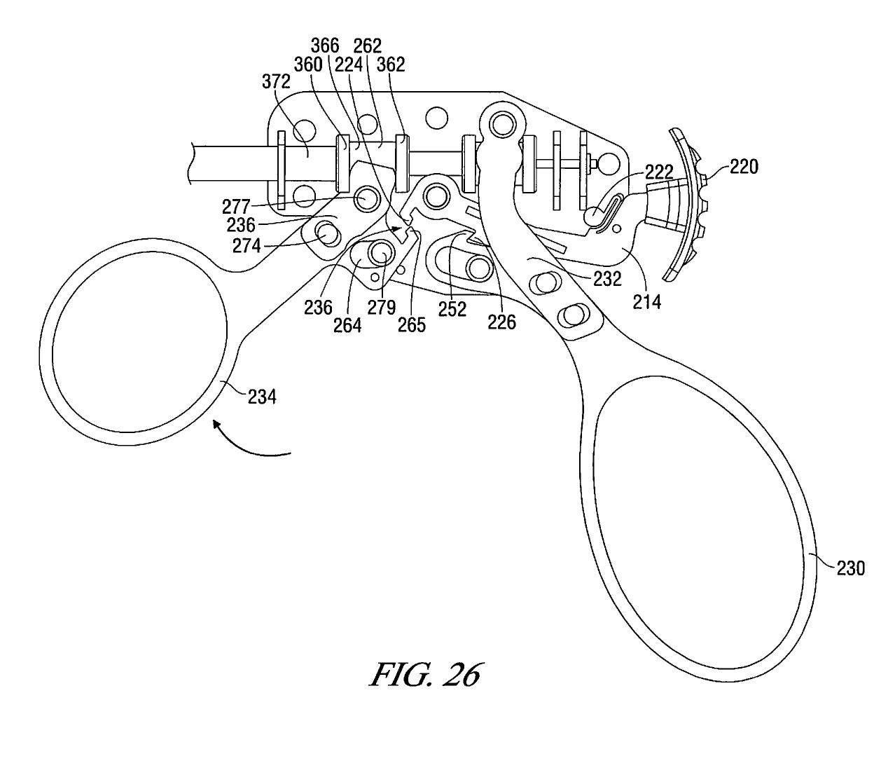

FIG. 26 is a side view similar to FIG. 25 showing movement of the cutter handle to open the jaws in a cutting function;

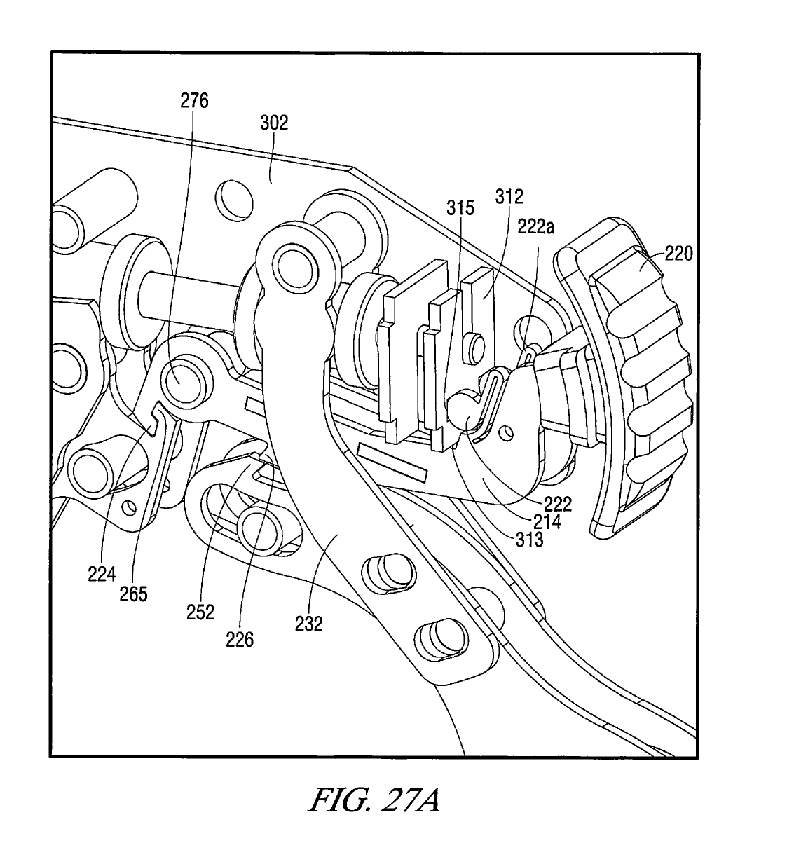

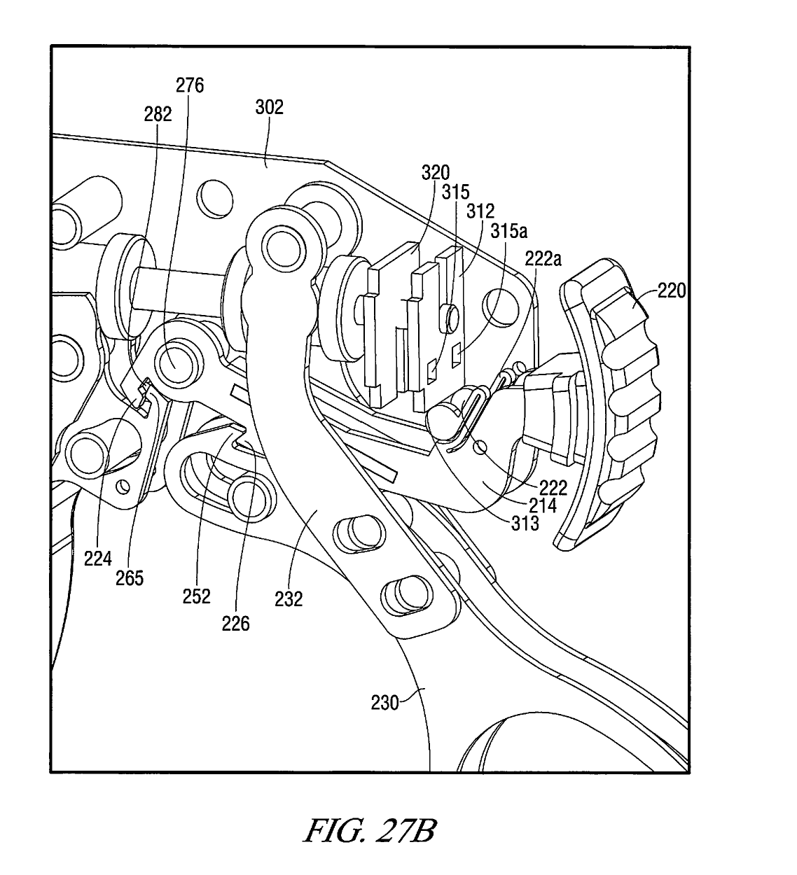

FIG. 27A is a close up view showing the selector retained in the grasping function position;

FIG. 27B is a close up view showing the selector retained in the cutting function position;

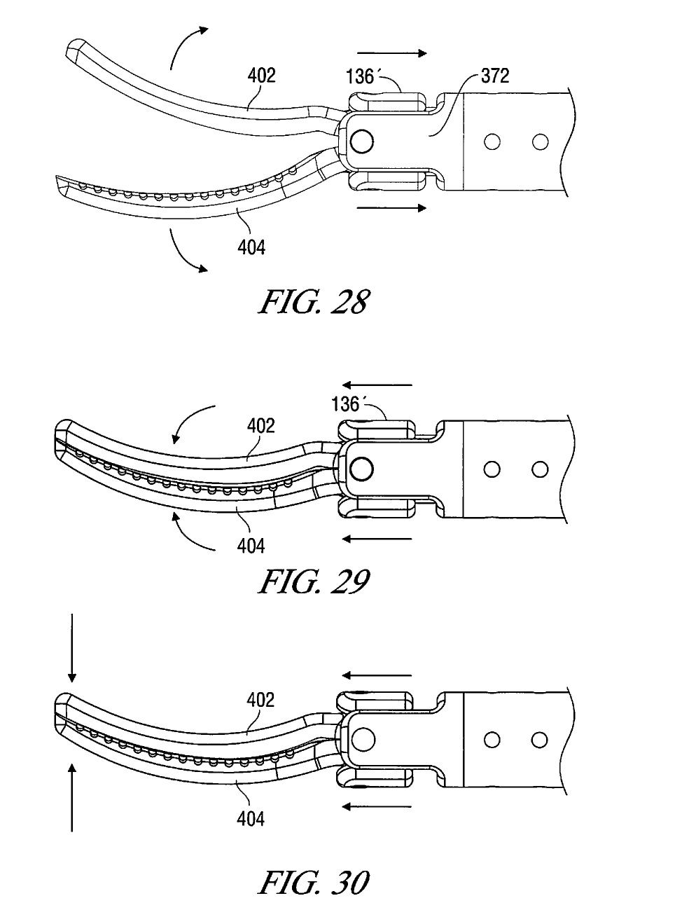

FIG. 28 is a side view of the jaws in the open grasping position corresponding to the handle position of FIG. 23;

FIG. 29 is a side view of the jaws in the closed grasping position corresponding to the handle position of FIG. 22;

FIG. 30 is a side view of the jaws in the tensioned position corresponding to the handle position of FIG. 24;

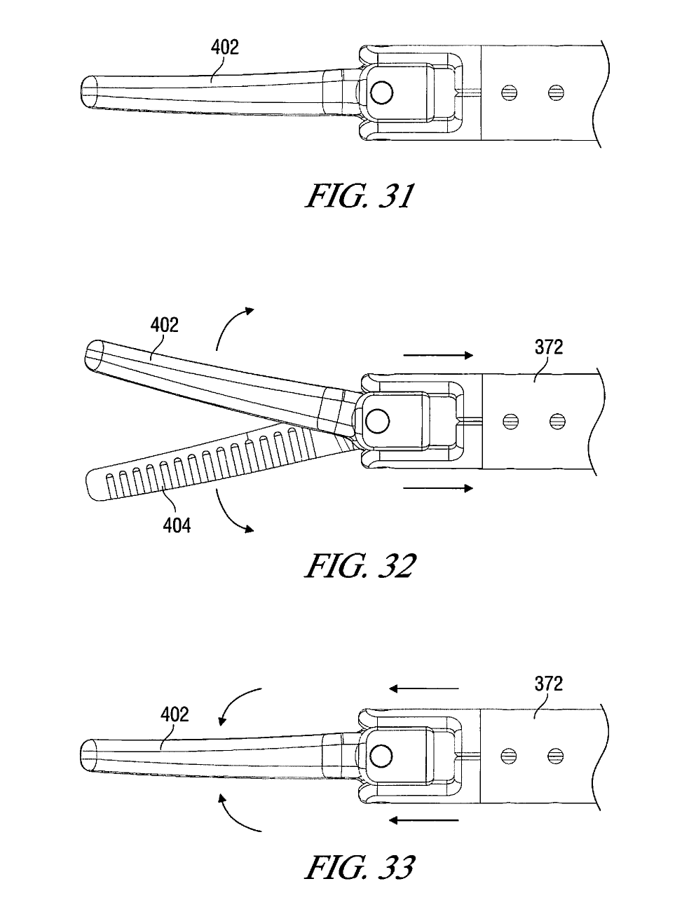

FIG. 31 is a top view of the jaws in the closed cutting position corresponding to the handle position of FIG. 25;

FIG. 32 is a top view of the jaws in the open cutting position corresponding to the handle position of FIG. 26;

FIG. 33 is a top view similar to FIG. 31 with arrows showing the jaws returned to the closed cutting position corresponding to the handle position of FIG. 25; and

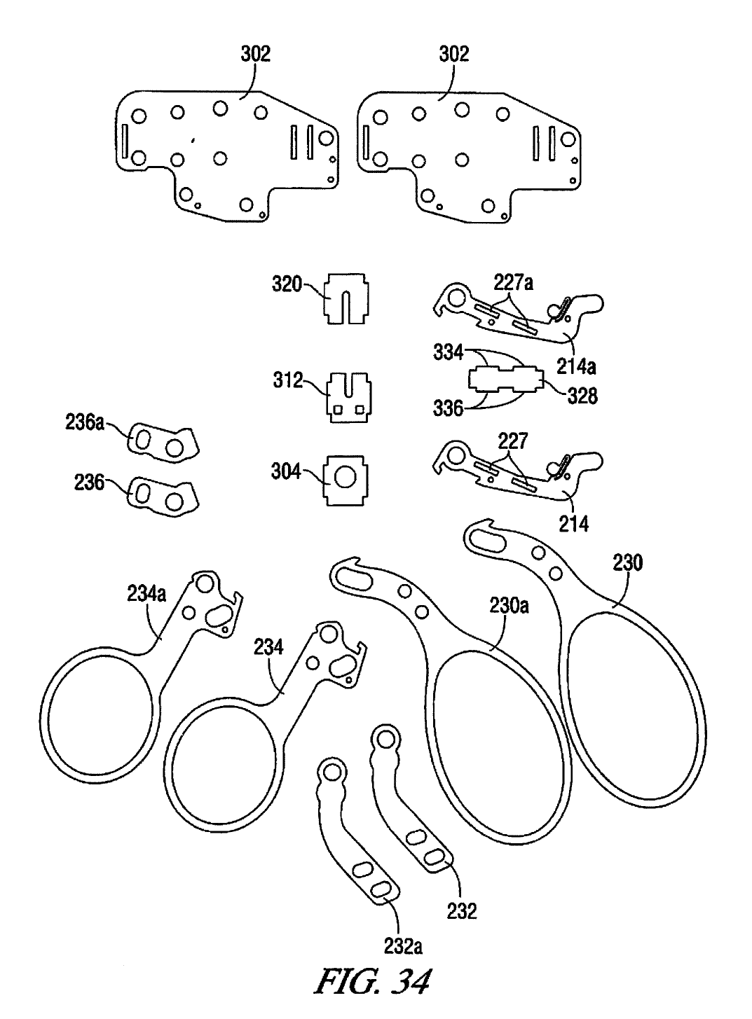

FIG. 34 is a side view showing components of the handle assembly formed from a metal sheet in accordance with one embodiment of the present invention.

### DETAILED DESCRIPTION OF PREFERRED EMBODIMENTS

Turning now to the drawings, wherein like reference numerals identify similar or like components throughout the several views, a first embodiment of the surgical instrument of the present invention is designated generally by reference numeral 10.

Throughout the description, the term “proximal” will refer to the portion of the instrument closer to the user and the term “distal” will refer to the portion of the instrument further from the user. Additionally, throughout the description, the terms “upper” and “lower” are used in relation to the position of the instrument in the drawings. Clearly, if the orientation of the instrument changes, the references to upper and lower will also change.

Turning first to FIG. 1, the surgical instrument 10 has a handle assembly 30 at its proximal portion 11, an elongated member or shaft 20 extending distally from the handle assembly 30 and a jaw assembly 100 at the distal portion 13. The jaw assembly 100 includes a first jaw 110 and a second jaw 120 which are operably connected to the handle assembly 30. In the orientation of FIG. 1, the first jaw 110 forms the lower jaw and the second jaw 120 forms the upper jaw. A rotating knob 26 adjacent the handle assembly 30 and at a proximal region of the elongated shaft (outer tube) 20 rotates the shaft 20 and thereby the jaws 110, 120 of the instrument 10 about a longitudinal axis of the shaft 20. A rocker switch 51 of switching mechanism 50 (FIG. 2) enables switching between a grasping and cutting function of the jaws 110, 120 as described in more detail below. A receptacle 24 for a conventional monopolar cautery plug optionally extends from the top of the body of handle assembly 30.

The jaws 110, 120 are movable in a first orientation to perform a cutting function and movable in a second different orientation to perform a grasping function. Thus, the jaws move in different planes to cut (sever) tissue and to grasp tissue. More specifically, in a grasping action, jaws 110 and 120 pivot about a first pivot axis in planes at acute angles to each other so the upper surface 112 of lower jaw 110 and the lower surface 124 of upper jaw 120 move away from each, forming an acute angle in the open position. Stated another away, the opposing edges or walls 110a, 110b of jaw 110 and opposing edges 120a, 120b of jaw 120 move downwardly and upwardly, respectively, away from each other in a grasping function. This is depicted in FIGS. 5 and 8 wherein the jaws 110, 120 are shown in FIG. 5 in a closed configuration and in FIG. 8 in an open grasping position.

In a cutting action, the first and second jaws 110, 120 pivot about a second pivot axis such that the upper surface 112 of jaw 110 and lower surface 124 of jaw 120 move away from each other in substantially parallel planes in a scissor-like action. In this manner, edge 110b of lower jaw 110 which has a sharpened surface 111 interacts with an edge of upper jaw 120 opposite edge 120b which has a sharpened surface. This is shown in FIGS. 11 and 13 wherein the jaws 110, 120 are shown in the open and closed positions, respectively. When moved to the closed position, the sharpened surface 111 on edge 110b of lower jaw 110 passes by the sharpened surface on the lower edge of upper jaw 120. (Note this sharpened edge of upper jaw 120 is not visible in FIG. 11 as it is opposite edge 120b; however the sharpened edge is shown in the upper jaw of the alternate embodiment of FIG. 14 discussed below).

As shown in FIG. 5, the first and second pivot axes, i.e., axis D about which the jaws 110, 120 move in the grasping orientation and axis A about which the jaws move in a cutting orientation, intersect a center point C and are preferably substantially perpendicular. By being substantially perpendicular, the axes intersect between about 80° to about 100°, but more preferably at about 85° to about 95° and more preferably at about 90 degrees.

Although both jaws are shown and described as moving in the cutting and grasping action, it is also contemplated that alternatively only one of the jaws could move in the grasping action and/or in the cutting action with the other jaw remaining fixed. Therefore, relative movement of the jaws as used herein refers to one of the jaws moving with respect to the other fixed jaw or both of the jaws 110, 120 moving between their various positions.

With reference to FIG. 3, a lower projecting member or pin 113 extends from the lower surface 114 of lower jaw 110 and is configured to engage opening 131b in yoke half 136b for movement of the jaw 110 in a grasping action (FIG. 8). A side projecting member or pin 115 engages the cutter tube 150 (described below) for movement of the jaw 110 in a cutting action (FIG. 11).

Lower jaw 110 has a proximal upper recess 119, preferably substantially hemispherical as shown to receive a ball 132 of shaft 130. The shaft 130 and ball 132 are held in tension by tension spring 133 (see e.g. FIG. 4) to remove the tolerances, i.e. reduce the play of the jaws. Jaw 110 also has a linear region 118a and an arcuate region 118b.

Upper jaw 120 is similar to lower jaw 110 and has an upper surface 122 opposite lower surface 124. An upper projecting member or pin 123 extends from the upper surface 122 and is configured to engage opening 131a in yoke half 136a for movement of the jaw 120 in a grasping action. A projecting member or side pin 125 engages the cutter tube 150 (described below) for movement of the jaws 110 in a cutting action. Note that side pins 115 and 125 extend from opposing sides of the jaw assembly 100. Preferably pins 113 and 123 lie substantially along the same axis and pins 115 and 125 lie substantially along the same axis.

Upper jaw 120 has a proximal lower recess similar to recess 119 of jaw 110, preferably substantially hemispherical, to receive ball 132 of shaft 130. Jaw 120 has a linear region 128a and an arcuate region 128b.

Preferably both jaws are movable, however, as noted above, it is also contemplated that one of the jaws could be fixed and the other movable. Also, other configurations of the jaws other than curved as shown are also contemplated. In the embodiment of FIG. 3, the jaws are shown with blunt tips.

Each of the jaws preferably has a series of teeth shown for example in the preferred alternate embodiment of FIG. 14. As shown, lower jaw 410 has teeth 411 on upper surface 413 and upper jaw 420 has teeth 421 on lower surface 427. Preferably the teeth 411, 421 extend substantially transversely to a longitudinal axis of the jaws 410, 420. The edge 415 of the lower jaw 410 has a raised rib 416 with a cutting surface. The upper jaw 420 has a raised rib 426 on its lower surface 427 of the edge 428 opposite edge 425 with a cutting surface to interact with the cutting surface of raised rib 416 when the jaws 410, 420 are moved in a scissor like fashion to sever tissue in the manner of FIGS. 11 and 13. The raised ribs 416 and 426 on the lower and upper jaws, respectively, illustratively extend beyond the plane of the respective teeth 411, 421 so as not to interfere with the scissors action of jaws 410, 420. In all other respects, including their configuration and actuation, jaws 410, 420 are identical to jaws 110 and 120 of FIG. 1.

The switching mechanism 50 for selectively locking the cutter or grasping gear mechanism of the instrument will now be described. With reference to FIGS. 2, 4, and 9, the switching mechanism 50 includes a rocker switch 51 positioned at a proximal portion of the instrument and having a pair of detents 51a engagable with a respective recess 33a or 33b in the housing 32 of handle assembly 30. A mounting pin 52a extends outwardly from each side of switch 51 and engages aperture 39a in housing half 32a and a similar aperture (not shown) in the other housing half 32b. Such mounting to the housing 32 provides for pivoting (rocking) movement of the switch 51. Note that only one of the housing halves, half 32a, is shown in FIG. 2; the other half 32b is not shown for clarity and is the mirror image of housing half 32a. Therefore only the mounting to housing half 32a will be described, it being understood that the components are mounted to housing half 32b in the same manner.

The detents 51a of switch 51 retain the rocker 51 in either a first position where the jaw grasping function is locked out or a second position where the jaw cutting function is locked out. This is achieved by engagement in either upper recess 33b of housing 32a (and housing 32b) for the cutting function or a lower recess 33a for the grasping function. The detents 51a can be retained in positions other than those shown e.g. a neutral position, and two or more detents can be provided and the detents can be provided in other parts of the switch. Other ways to retain the switch in its positions could also be provided. The switch 51 can include a curved indentation 54 which can have a knurled or irregular surface to facilitate actuation by the surgeon's thumb.

The switching mechanism 50 further includes a linkage mechanism operably associated with the rocker switch 51, best shown in FIGS. 2, 4 and 9. More specifically, the linkage mechanism includes a switch connecting link 56, a pair of rear (first) intermediate links 60a, 60b, a front (second) intermediate link 64, and a curved gear link 70. Link 56 is connected to an upper extension portion of rocker 51 via pin 56. Rear intermediate links 60a, 60b each have a pin for attachment to a front end of link 56. Rear intermediate links 60a, 60b are attached to a rear portion of front intermediate link 64 via pin 64b extending through opposing openings in link 64. Pin 64b is mounted within opening 36c in housing half 32a (as well as the other housing half 32b). The front intermediate link 64 has front holes aligned with the holes on extension 72 of curved link 70 for attachment to curved link 70 via pin 64a. Note in FIGS. 2, 4 and 7, the switching mechanism is in the grasping function position. In FIGS. 9 and 12, the switching mechanism is in the cutting function position.

A safety mechanism 160 is preferably provided to ensure the switching mechanism 50 cannot be activated unless the jaws 110, 120 are in the closed position. This is described in more detail below in connection with the alternate embodiment of FIG. 15.

Curved gear link 70 has a pair of rear holes and a pair of front holes to receive gear pins 77, 79, respectively. Central mounting holes 73 of link 70 receive housing pins 35 on each housing half 32a, 32b (or alternatively a single pin trapped between the housing halves) for movably (pivotably) mounting curved gear link 70 to the housing 32.

Curved link 70 moves in a rocker type fashion, pivoting about housing pin 35 connected in holes 73, preferably centrally disposed in link 70, to selectively engage one of the gear mechanisms. In this rocking movement, the curved link 70 moves between one position to effect engagement of the grasper gear mechanism and another position to effect engagement of a cutter gear mechanism, both described below.

The gear mechanism of the present invention provides a system for advancing the respective actuator tubes for opening and closing the jaws in a grasping or in a cutting function. The gear mechanism is also selectively lockable to lock movement of one of the actuator handles and respective actuator tubes while enabling movement of the other actuator handle and respective tube.

With continued reference to FIGS. 2, 4 and 7, the gear mechanism includes an upper grasper spur gear 82, a lower grasper inverted spur gear 85 and a grasper gear 84. The gear mechanism further includes an upper cutter spur gear 86, an inverted cutter spur gear 89 and cutter gear 88 positioned distally of the grasper gears 82, 84, and 85. As shown, preferably the teeth 84a, 88a of gears 84, 88 extend circumferentially.

The handle assembly is operably associated with the gears 84 and 88 such that movement of one of the handles moves the respective gears which in turn actuates a connected movable member, e.g. an actuator tube or shaft, to open and close the jaws. This is described in more detail below in connection with the discussion of the handle assembly.

The spur gears 82 and 86 of the gear mechanism 80 form locking mechanisms to selectively lock the respective gear 84, 88 against movement. In this manner, when a cutting action is desired, the rocker switch 51 is activated, i.e. manually actuated by the user to pivot about pivot pin 52a, to move the links of the switching mechanism 50 so that the teeth 82a of the grasper spur gear 82 mesh with the teeth 84a of grasper gear 84 to lock the grasper gear 84 against movement. This prevents movement of the grasper handle actuator 40 and is shown in FIGS. 9 and 12. When a grasping action is desired, the rocker switch 51 is activated to move the links of the switching mechanism 50 so that the teeth 86a of the cutter spur gear 86 mesh with the teeth 88a of the grasper gear 88 to lock the grasper gear 88 against movement. This prevents movement of the cutter handle actuator 46 and is shown in FIGS. 4 and 7.

More specifically, when the lower portion 51c of rocker switch 51 is pivoted toward the housing 32, link 56 is pulled back to pivot about pin 56b to a position more aligned with a longitudinal axis of the instrument as shown in FIG. 9. This in turn pivots rear intermediate links 60a, 60b to a position more aligned with the longitudinal axis of the instrument, thereby pulling front intermediate link 64 to pivot the rear portion of curved link 70 (via the connection pin 64a) downwardly so that cutter spur gear 86 is disengaged from cutter gear 88 and grasper spur gear 82 is engaged with grasper gear 84 to lock the gear 84 against sliding movement.

When the upper portion 51b of rocker switch 51 is pivoted toward the housing 50, link 56 is pivoted about pin 56b to a more angled position as shown in FIG. 4. This in turn pivots rear intermediate links 60a, 60b to a more angled position, thereby forcing a distal portion of front intermediate link 64 to move downwardly to pivot curved link 70 (via the connection pin 64a) counterclockwise so that grasper spur gear 82 is disengaged from grasper gear 84 and cutter spur gear 86 is engaged with cutter gear 88 to lock the gear 88 against sliding movement.

Turning now to the movable elements which operably connect the gears with the jaws, a grasper tube 140 and a cutter tube 150 are provided. More specifically and with reference to FIGS. 2, 3, 4 and 7, extending from grasper gear 84 is grasper tube 140. Grasper tube 140 is attached to yoke 136 (formed from yoke halves 136a, 136b) at its distal end 140a (see FIG. 3). When the grasper handle 40 is actuated to perform the grasping function, i.e., move the jaws 110, 120 from a closed position to an open position in the grasping orientation, grasper gear 84 is moved proximally from its distal position of FIG. 4 to its proximal position of FIG. 7, pulling attached grasper tube 140 and attached yoke 136 proximally. This moves the jaws 110, 120 from the closed position of FIG. 5 to the open grasping position of FIG. 8 due to the engagement of respective upper and lower pins 123, 113 of upper and lower jaws 120, 110 with respective openings 131a, 131b in yoke 136. That is, this pin engagement causes the jaws to pivot about axis “D” transverse to the longitudinal axis “B” of the jaws and instrument and passing through the side pins 125, 115. To close the jaws 120, 110, the grasper handle 40 is moved in the opposite direction (distally) back to its normal at rest position, thereby moving grasper gear 84 distally to thereby advance the grasper tube 140 and attached yoke 136 distally, forcing the jaws 120, 110 to pivot about axis “D” back to the position of FIG. 5. Note that outer tube (elongated member) 20 has slots 20a (FIG. 3) to accommodate pins 123, 113.

The grasper tube 140 is positioned inside and preferably coaxially with the cutter tube 150. Cutter tube 150 is slidably mounted within recesses formed in ribs 31 of housing half 32a and corresponding ribs on housing half 32b. (see FIG. 2). Cutter tube 150 is positioned within shaft 20.

Referring to FIGS. 3, 9 and 12, cutter tube 150 has a proximal end extending from cutter gear 88 and a distal end 150a. The distal end 150a has holes 152a, 152b on respective extensions 153a, 153b to receive side pins 115, 125 of jaws 110, 120, respectively. When the cutter handle 46 is actuated to perform the cutting function, i.e. move the jaws 110, 120 from a closed position of FIG. 13 outwardly to an open position of FIG. 11 in the cutting orientation, cutter gear 88 is moved proximally, pulling cutter tube 150 proximally from its distal position of FIG. 9 to its proximal position of FIG. 12. This moves the jaws 110, 120 to an open position due to the engagement of the holes 152a and 152b of extensions 153a, 153b with the side pins 115, 125. This causes the jaws 110, 120 to pivot about a cutting axis “A” extending transverse to the longitudinal axis of the jaws and instrument and passing through the upper and lower pins 123, 113. To close the jaws 110, 120 to cut tissue, the cutter handle 46 is moved in the opposite direction (proximally) to return to its at rest position, thereby moving cutter gear 88 distally to thereby advance the cutter tube 150 distally to force jaws 120, 110 back to the closed position of FIG. 13. Note that outer tube (elongated member) 20 has slots 20b (FIG. 3) to accommodate pins 125, 115.

Shaft 130 has a ball 132 at its distal end 131, preferably integral therewith, and configured and dimensioned to fit within the hemispherical recesses (e.g. recess 119) of jaws 110, 120, respectively. The hemispherical recesses together form a spherical recess. Shaft 130 is supported within a recess in rib 31b in housing half 32a and a corresponding rib in housing half 32b (see FIG. 2).

Turning now to the handle assembly 30 and with initial reference to FIGS. 1 and 2, the assembly 30 includes first and second body halves 32a, 32b fastened together by conventional methods. The handle assembly 30 has a stationary handle or grip 34 with finger loop 28, a movable grasper handle or actuator 40 with finger loop 42, and a cutter (scissor) handle or actuator 46 with finger loop 48. As shown, grasper (grasping) handle 40 is positioned proximally of stationary handle 34 and cutter (cutting) handle 46 is positioned distally of stationary handle 34. Grasper handle 40 is operably associated with the grasper gear 82 and cutter handle 46 is operably associated with cutter gear 86. The body halves have slots to receive the mounting portions of the handles and to accommodate movement of the handles 40, 46.

Referring to FIGS. 2 and 4, yoke 43 of grasper handle 40 has spaced openings 43a for mounting to handle pin 37a of housing 32a and a corresponding handle pin on housing half 32b. Yoke 47 of cutter handle 46 has spaced openings 43a for mounting to handle pin 38a of housing 32a and a corresponding handle pin on housing half 32b.

Grasper handle 40 has an internal cam slot 49 which is configured to receive lower gear pin 85a extending from a lower portion of lower grasper spur gear 85. In this manner, when grasper handle 40 is retracted, i.e., moved in a proximal direction from the position of FIG. 4 to the position of FIG. 7, the cam slot 49 forces lower gear pin 85a proximally to retract grasper gears 85 and 84 proximally. This retracts the attached grasper tube 140 to open the jaws 110, 120 in a grasping fashion described above. This movement can be appreciated by comparing FIGS. 4 and 7—FIG. 4 corresponding to the position of the jaws 110, 120 in the closed position of FIG. 5; and FIG. 7 corresponding to the position of the jaws 110, 120 in the open grasping orientation of FIG. 8. Movement of the grasper handle 40 distally back to its initial at rest position cams the lower gear pin 85a distally to move the grasper gear 84 distally back to its original position, thereby moving the grasper tube 140 distally to pivot the jaws 110, 120 back to the closed position. Note that as shown in FIG. 4, the lower gear pin 85a is below and distal of the pivot axis (extending through openings 43a) of grasper handle 40.

Referring to FIG. 9, cutter handle 46 has an internal cam slot 41 which is configured to receive lower gear pin 89a extending from lower grasper inverted spur gear 89. In this manner, when cutter handle 46 is moved in a distal direction, the lower gear pin 89a is cammed proximally to move the lower cutter spur gear 89 and cutter gear 88 proximally. This retracts the attached cutter tube 150 to open the jaws 110, 120 in a cutting fashion described above. This movement can be appreciated by comparing FIGS. 9 and 12—FIG. 9 corresponding to the position of the jaws 110, 120 in the closed position of FIG. 13; and FIG. 12 corresponding to the position of the jaws 110, 120 in the open cutting orientation of FIG. 11. Movement of the cutter handle 46 proximally back to its initial at rest position cams the lower gear pin 89a distally to move the cutter gear 88 distally back to its original position, thereby moving the cutter tube 150 distally to pivot the jaws 110, 120 back to the closed position. Note in the position of FIG. 7, lower gear pin 89a is above and distal of the pivot axis (extending through openings 47a) of cutter handle 46.

The handle assembly provides an ergonomic handle design for moving the jaws as well as a built in safety. This is depicted in FIGS. 1, 6 and 10 which illustrate a surgeon's hand engaging the handle assembly 40. As shown in FIG. 1, both the grasping handle loop 42 and the cutting handle loop 48 of handles 40, 46, respectively, are in the at rest position so that the jaws 110, 120 are closed. The user's thumb is disengaged from grasper loop 42 to access switch 51 on housing 30. The switch 51 is in the grasping function position.

If the user desires to perform a grasping function, the instrument is held as shown with the thumb of the user through grasper loop 42 and the forefinger remaining through the cutter loop 48 (alternatively the forefinger can be removed from the cutter loop 48 during the grasping actuation). The middle and ring finger extend through the handle loop 28 of stationary handle 34. To open the jaws to grasp tissue, the user with his/her thumb moves the grasper handle 40 away from the stationary handle 34 in the direction of the arrow of FIG. 6. This moves the jaws 110, 120 to the open grasping position of FIG. 8. The user then moves the grasper handle 40 distally. i.e., back towards the stationary handle 34 to move the jaws 110, 120 toward each other to grasp tissue therebetween. The jaws 110, 120 move such that the top surface of the bottom jaw 110 and the bottom surface of the top jaw 120 move toward each other in a pivoting fashion.

To switch to the cutting function, the instrument 10 is grasped as shown in FIG. 10 with the thumb of the user removed from the grasper loop 42 to access switch 51. Switch 51 switches the linkage mechanism of the instrument between a grasping and a cutting function such that only one function can be operable at a time as described in detail above. Switch 51 can be activated by the single hand of the user only when the jaws are in the closed position. The ergonomic design of the handle achieves this as: 1) the thumb of the user needs to be removed from the grasper loop 42 to access the switch 51 so that the grasper handle 40 can no longer be held in its outward (open) position to open the jaws; and 2) to reach the switch 51 with the thumb, the grasper handle 40 is blocked in the inward (closed) position by the palm of the user's hand as shown in FIG. 10. A safety mechanism can also be provided to ensure the switch 51 can be activated only when the jaws 110, 120 are closed. This is described below in conjunction with the embodiment of FIG. 15.

After the switch 51 is rotated to the cutting position by pressing the lower portion 51c of the switch 51, it actuates the linkage mechanism as described above to lock the grasper gear 84 and grasper handle 40 to thereby lock movement of the jaws 110, 120 in a grasping function. To effectuate cutting, the forefinger of the user remains in the cutter loop 48 of cutter handle 46 and the thumb remains either outside the grasper loop 42 of grasper handle 40, resting on the rear of housing 32 as shown in FIG. 1 or inside the lop 42 as in FIG. 6. The remaining fingers are also in the same position as in the grasping and switching function, i.e., the middle and ring fingers extending through the handle loop 28 of stationary handle 34. A groove 31 can optionally be provided on stationary handle 34 for resting of the small finger.

To open the jaws in a scissor like fashion to cut/sever tissue, the user with his/her forefinger moves the cutter handle 46 away from the stationary handle 34. This moves the jaws 110, 120 to the open cutting position of FIG. 11. To close the jaws 110, 120, the grasper loop 46 is moved back towards the stationary handle 34. Note, as described above, in the cutting function, the jaws 110, 120 move such that the inner edges of the jaws move toward/across each other in a scissor like fashion. This can be appreciated by comparing FIGS. 11 and 13.

If the user wants to return to the grasping function, the user grasps the handle assembly 30 in the manner shown in FIG. 1 and presses the upper portion 51b of switch 51 to pivot the switch 51 from the position of FIG. 10 to the position of FIG. 1. This actuates the linkage mechanism to lock the cutter gear 88 and cutter handle 46 as described above (and releases the grasper spur gear lock on the grasper gear 84). The user can then grasp the instrument as shown in FIG. 6 to move the jaws 110, 120 in a grasping function. As can be appreciated, the instrument is designed so it can't be switched with the grasping hand of the user between cutting and grasping functions unless the jaws 30 are in the closed position.

In addition or as an alternative to the ergonomic design having this built in safety, a safety mechanism 160 can also be provided. This is shown in the alternative embodiment of FIGS. 15 and 16. The embodiment of FIGS. 15 and 16 is identical to the embodiment of FIG. 1 except for the safety mechanism and the apertures in the switch. Therefore, the actuator handles, links, etc. have not been labeled for clarity. Corresponding parts with the FIG. 1 embodiment discussed in conjunction with the safety mechanism 160 have been given “prime” designations.

The safety mechanism 160 includes a slidable locking plate 171 having a post 172 extending from a proximal portion 174. Spring 176 biases the locking plate 172 in a distal direction. Inner tab 178 at the proximal portion 174 abuts or engages the rear wall of the grasper gear 84′ and inner tab 179 at the distal portion 175 of plate 171 abuts or engages the rear wall of the cutter gear 88′. In the closed position of the jaws 110, 120, both grasper gear 84′ and cutter gear 88′ are in the forward position. In this position, the locking plate is biased distally by spring 176, out of engagement with switch 180.

If the jaws are in the open cutting position, cutting gear 88′ is in the retracted position of FIG. 16, which moves locking plate 171 to a retracted position against the force of spring 176. In this retracted position, the post 172 is positioned in a lower aperture 181a of the switch 180. In this position, the switch 180 cannot be moved as its pivotable movement is blocked. Similarly, if the jaws are in the open grasping position, (with the switch 51 in the grasping function position with the upper portion 51b closer to the housing 32) grasper gear 84′ is in the retracted position, which moves locking plate 171 to a retracted position against the force of spring 176. In this retracted position, the post 172 is positioned in an upper aperture 181b of the switch 51 to prevent pivotable movement of the switch 180. Note besides the apertures 181a, 181b, switch 180 is identical to switch 51 described above.

The use of the instrument will now be described. For purposes of this description, the instrument is packaged with the switch 51 in the grasping function position so it's initially ready for grasping; however, the instrument can alternatively be packaged with the switch 51 in the cutting function position. Note that the switch 51 preferably includes indicia on its rear surface so the user has a visual indication of which function the switch 51 is engaged. Also, throughout the description of use, reference is made to how the safety mechanism 160 of FIGS. 15 and 16, if utilized, would function.

In the initial position of the instrument 10 shown in FIG. 1, the grasper handle 40 is in the forward (distal) position, spaced closer to the stationary handle 34 so that jaws 110 and 120 are in the closed position of FIG. 5. The cutter handle 46 is in the retracted (proximal) position, closer to the stationary handle 34. The instrument 10 is inserted through an access port or opening with the jaws 110, 120 closed.

In this initial position, the switch 51 is in the grasping position such that upper portion 51b is angled toward the handle housing 30 and the lower portion 51c is angled away from the housing 32. In this position, shown in FIG. 4, rocker engaging link 56 is angled upwardly (in the orientation of FIG. 4), causing rear intermediate links 60a, 60b, to be angled upwardly. This results in front intermediate link 64 applying a force to curved link 70 so the curved link 70 is pivoted forwardly (counterclockwise) about central housing pin 73 toward cutter gear 88 to move teeth 86a of cutter spur gear 86 into engagement with the teeth 88a of cutter gear 88. Consequently, this intermeshing of the teeth 86a and teeth 88a locks gear 88 so movement is prohibited. Thus, the user cannot move cutter handle 46 and cannot advance cutter actuator 150. With the locking of cutter handle 46, the user is prevented from confusing the cutting and grasping function. Note that the teeth 82a of the grasper spur gear 82 in this position are spaced (disengaged) from the teeth 84a of the grasper gear 84 to allow movement of gear 84.

Note also in the closed position of the jaws 110, 120, the cutter gear 88 and grasper gear 84 (and lower spur gears 85, 89) are in the forward position so that in the embodiment of FIGS. 15 and 16 utilizing safety mechanism 160, safety plate 171 is in the forward position. In this forward (disengaged) position, the proximal post 172 of safety plate 171 is disengaged from the switch 180 and is spaced distally from the apertures 181a, 181b to enable pivoting movement of switch 180.

The instrument 10 is inserted with the jaws 110, 120 closed through an access port or opening and advanced toward the surgical site. If the surgeon desires to use the grasping function, the user retracts grasper handle 40 in the direction of the arrow of FIGS. 6 and 7, moving it away from the stationary handle 34. When the grasper handle 40 is moved in this direction, handle 40 pivots about handle mounting pin 37a (FIG. 2) and forces gear 84 proximally via engagement of grasper lower gear pin 85a of attached lower spur gear 89 in cam slot 49 (FIG. 7). This causes retraction of grasper tube 140 to retract attached yoke 136 which pivots the jaws 110, 120 about pivot axis D (FIG. 5) in the grasping orientation from the closed position to the grasping open position of FIG. 8 via the engagement of upper and lower jaw pins 123, 113.

Note that in this retracted position of the grasper gear 84, if the safety mechanism 160 of FIG. 15 is utilized, safety plate 171 is likewise retracted (due to its abutment with the back of the grasper gear 84). In this retracted position, post 172 is positioned within aperture 181b of switch 180. This prevents movement of switch 51 so the user cannot switch from the grasping function to the cutting function if the jaws 110, 120 are in the open position.

To close the jaws in this grasper function to grasp tissue positioned between the jaws 110, 120, grasper handle 40 is moved in the opposite direction (distally toward stationary handle 34) thereby moving grasper gear 84 via lower gear pin 85 distally to advance grasper tube 140 so jaws 110, 120 can pivot about pivot axis D back to the closed position of FIG. 5.

If the surgeon desires to switch to the cutting function of the instrument 10, the user pivots switch 51 to its cutting function position by pressing lower portion 51c toward housing 32 so that the lower portion 51c is closer to handle housing 32 and the upper portion 51b is positioned further away from housing 32 as shown in FIG. 10. Note that detents 51a of switch 51 are moved from engagement with upper slot 33b in housing half 32a (FIG. 2) into engagement with lower slot 33a of housing half 32a (and a corresponding slot in housing half 32b) to provide a tactile indicator that the switch 51 is in the cutting position as well as to maintain the switch 51 in this position so the user does not need to hold the switch 51.

Such pivoting motion of switch 51 pulls attached link 56 proximally, which in turn pulls rear intermediate link 60a 60b proximally to the more linear position of FIG. 9. This forces front intermediate link 64 to pivot curved link 70 in a clockwise direction about central mounting pin 73. This clockwise movement lifts cutter spur gear 86 out of locking engagement with cutter gear 88 and moves grasper spur gear 82 into locking engagement with grasper gear 84. In this grasper locking position, teeth 82a of spur gear 82 intermesh with teeth 84a of grasper gear 84 so that movement of the grasper gear 84 is prohibited. Thus, the user cannot move grasper handle 40 to actuate grasper tube 140.

Note again that if the safety mechanism 160 of FIGS. 15 and 16 is used, in the closed position of the jaws 110, 120, the safety plate 171 is in the forward position as both the cutter gear 88 and grasper gear 84 are in the forward position. In this forward (disengaged) position, the proximal post 172 of safety plate 171 is disengaged from the apertures 181a, 181b of switch 180 as it is spaced distally from the apertures.

To move jaws 110, 120 in a cutting function, the user moves cutter handle 46 distally, moving it in a direction away toward stationary handle 34 as shown in FIG. 12. When the cutter handle 46 is moved in this direction, cutter handle 46 pivots about handle mounting pin 38a (FIG. 2) and cutter lower gear pin 89a of lower inverted spur gear 89 (attached to cutter gear 88) is cammed rearwardly by internal cam slot 47 of cutter handle 40. This causes retraction of cutter gear 88 which retracts attached cutter tube 150. Retraction of cutter tube 150 pivots the jaws 110, 120 about pivot axis A in the cutting orientation to an open cutting position as side pins 115 and 125 are engaged by extensions 153a,153b of cutter tube 150 (see FIG. 11).

Note that in this retracted position of the cutter gear 86, safety plate 171, if provided, is likewise retracted (due to its abutment with the back of the cutter gear 86). In this retracted position, post 172 is positioned within aperture 181a of switch 180. This prevents movement of switch 180 so the user cannot switch from the cutting function to the grasping function if the jaws 110, 120 are in the open position.

To close the jaws 110, 120, the cutter handle 46 is moved proximally toward the stationary handle 34 back to its at rest position, thereby advancing the cutter gear 86 and cutter tube 150 distally.

As can be appreciated, if the user desires to switch from the cutting function to the grasping function, switch 51 is pivoted so upper portion 51c is pressed toward the housing 32. As noted above, the jaws 110, 120 need to be in the closed position to activate the switch 51 because of safety plate 171. Detents 51a are moved from upper recess 33b to upper recess 33a of housing 32, again providing a tactile indicator that the switch 51 is in position. Such pivoting motion of the switch 51 forces links 56 and 60a, 60b, to the angled position of FIG. 4, causing intermediate link 64 to rotate curved link 70 in a counterclockwise direction to lift grasper spur gear 82 out of engagement with grasper gear 84 and to move cutter spur gear 86 into locking engagement with cutter gear 88. Thus, as described above, cutter handle 46 is locked and cannot be moved and the jaws 110, 120 can be opened in a grasping function. Safety plate 171 is out of locking engagement with switch 51.

As can be appreciated, the user can activate the switch to choose between the cutting and grasping functions as often as desirable. Thus, for example, in a laparoscopic cholecystectomy procedure, the surgeon can use instrument 10 to dissect tissue with the open jaws 110, 120, sever the duct with the jaws 110, 120 and then grasp the gall bladder with jaws 110, 120, avoiding the need for multiple instruments and instrument exchanges.

FIGS. 17-34 illustrate an alternate embodiment of the surgical instrument of the present invention. The surgical instrument is designated generally by reference numeral 200. Instrument 200 is similar to instrument 10 described above in that it includes a pair of jaws 402, 404 that are movable in a first orientation to perform a cutting function and movable in a second different orientation to perform a grasping function. That is, the jaws 402, 404 move in different planes in the same manner as described above with respect to jaws 110, 120 to cut (sever) tissue and to grasp tissue wherein a) in a grasping action, the jaws 402, 404 pivot about a first pivot axis in planes at acute angles to each other so the upper surface of the lower jaw and the lower surface of the upper jaw move away from each, forming an acute angle in the open position and b) in a cutting action, the first and second jaws 402, 404 pivot about a second pivot axis such that the upper surface of one jaw and the lower surface of the other jaw move away from each other in substantially parallel planes in a scissor-like action. Such movement of the jaws about such intersecting axes is described in detail with respect to jaws 110, 120 and such discussion of jaws 110, 120 is fully applicable to jaws 402, 404 of the embodiment of FIG. 17.

The surgical instrument 200, however, differs from surgical instrument 10 in several ways including the handle assembly and mechanisms to effect jaw movement and switching between grasping and cutting functions. Instrument 200 also differs from surgical instrument 10 in that it provides a mechanism to tension the jaws prior to movement in the cutting action to thereby enhance the cutting function.

It should be noted that as with the foregoing embodiments, although both jaws are shown and described as moving in the cutting and grasping action, it is also contemplated that alternatively only one of the jaws could move in the grasping action and/or cutting action with the other jaw remaining fixed. Therefore, relative movement of the jaws or effecting movement of the jaws as used herein refers to one of the jaws 402, 404 moving with respect to the other fixed jaw or both of the jaws 402, 404 moving between their various positions.

Turning now to details of the instrument 200 and with initial reference to FIG. 17, the surgical instrument 200 has a handle assembly 202 at its proximal portion 201, an elongated member or shaft 204 extending distally from the handle assembly 202 and a jaw assembly 400 at the distal portion 203, distal of elongated shaft 204. Handle assembly 202 includes a handle housing 207 (preferably composed of plastic) and a stationary handle 238 with a finger loop 239 and finger rest 237, a grasper handle or trigger 230 and a cutting (or scissor) handle or trigger 234. The jaw assembly 400 includes a first jaw 402 and a second jaw 404 which are operably connected to the handle assembly 202. In the orientation of FIG. 17 (and FIGS. 28-33), the first jaw 402 forms the upper jaw and the second jaw 404 forms the lower jaw. A rotating knob 206 adjacent the handle housing 207 and at a proximal region of the elongated shaft (outer tube) 204 rotates the shaft 204 and thereby the jaws 402, 404 of the instrument 200 about a longitudinal axis of the shaft 204. A switching mechanism 212 (FIG. 18A) enables switching between a grasping function and a cutting function of the jaws 402, 404 as described in more detail below. A receptacle 205 for a conventional monopolar cautery extends from the handle housing 207. A conductive spring 211 (FIG. 18B) provides electrical connection from the cautery receptacle 205 to the outer shaft 204 to provide electrical energy to the jaws 402, 404 for cauterizing tissue if desired. Note the spring 211 is preferably positioned at an angle for frictional contact to enhance the electrical connection. It is also contemplated that the instrument can be designed for bipolar as well as monopolar use.

The elongated shaft 204 forms a cutter tube 372 ie., tube that effects the jaw cutting action. A grasper tube 370, i.e., tube that effects the jaw cutting action. (FIG. 19) is coaxially slidably positioned within cutter tube 372. Cutter tube 372 and grasper tube 370 are operatively connected to the jaws 402, 404 in the same manner as cutter tube 150 and grasper tube 140 of the FIG. 1 embodiment so that axial movement of the tubes 372, 370 effect jaw movement, respectively, in a cutting action or grasping action. Note in an alternate embodiment, the elongated shaft and cutter tube are separate components, with the cutter tube coaxially slidably positioned within the stationary elongated shaft and the grasper tube coaxially slidably positioned within the cutter tube.

With reference to FIG. 21 which shows a perspective exploded view of components of the handle assembly 202, the handle assembly 202 includes a pair of grasper (or grasping) handles 230, 230a with finger loops 231, 231a, respectively, a pair of grasper links 232, 232a, a pair of scissor (or cutting) handles 234, 234a with finger loops 235, 235a, respectively, and a pair of scissor links 236, 236a. The grasper handles 230, 230a are connected together, e.g., attached to a plastic housing, and operate as one unit (see FIGS. 17 and 18A) and therefore when discussing movement herein will sometimes collectively be referred to as the grasper handle 230. Similarly, scissor handles 234, 234a are connected together, e.g., attached to a plastic housing, and operate as one unit and therefore when discussing movement herein will sometimes collectively be referred to as the scissor handle 234.

Handle assembly 202 further includes a pair of selectors or rockers 214, 214a which enable the user to select between a grasping function and a cutting function. The selectors 214, 214a are connected together and operate as one unit and therefore when discussing movement herein to the selected position will sometimes collectively be referred to as the selector 214. It should also be appreciated that in alternate embodiments instead of a pair, a single selector, grasper handle and/or scissor handle could be provided.

The pair of grasper handles 230, 230a are preferably identical and move as a unit with respect to stationary handle 238. Similarly, the pair of scissor handles 234, 234a are preferably identical and move as a unit with respect to stationary handle 238 (not shown in FIG. 21 for clarity). Grasper handle links 232, 232a are preferably identical, with grasper link 232 connected to grasper handle 230 on one side of instrument 200 and grasper link 232a connected to grasper handle 230a on the opposing side of the instrument 200. Similarly, scissor handle links 236, 236a are preferably identical, with scissor link 236 connected to scissor handle 234 on one side of instrument 200 and scissor link 236a connected to scissor handle 234a on the opposing side of the instrument 200.

Selectors 214, 214a are preferably identical and are positioned and attached within opening 221 of housing or button 220. The button can include a roughened or textured surface to enhance manipulation, e.g., sliding by the user. The selectors 214, 214a pivot about selector pin 215 (FIGS. 18A, 18B) which extends within openings 217, 217a of selectors 214, 214a. Selector 214 is engageable with scissor handle 234 and grasper handle 230 on one side of the instrument 200 and selector 214a is engageable with scissor handle 234a and grasper handle 230a on the opposing side of instrument 200. The selectors 214, 214a each include a biasing retaining spring 222, 222a, respectively, which retains the selectors 214, 214a in the selected position as described below. The selector 214 includes a distal latch or hook 224 forming an engagement or locking (blocking) surface for locking cutting action and a more proximal latch or hook 226 forming an engagement or locking (blocking) surface for locking grasping action. Similarly, the selector 214a includes a distal latch or hook 224a forming an engagement or locking (blocking) surface for locking cutting action and a more proximal latch or hook 226a forming an engagement or locking (blocking) surface for locking grasping action. The scissor locking surfaces 224, 224a and grasper locking surfaces 226, 226a are discussed in more detail below.

Grasper handle 230 has a finger loop 231 and a curved extension 240 forming a goose-neck shape. A slot 244 to receive pin 246 (FIG. 18A) is formed in the curved extension 240. Openings 248, 250 receive pins 255, 253 (FIG. 19). Similarly, grasper handle 230a has finger loop 231a and a curved extension 240a forming a goose-neck shape with a slot 244a to receive pin 246. Openings 248a, 250a receive pins 255, 253. The grasper handle link 232 is attached to the grasper handle 230 with pin 255 extending through upper slot 256 and pin 253 extending through lower slot 258 (see FIG. 19). The second grasper handle link 232a is attached to grasper handle 230a with pin 255 extending through upper slot 256a and pin 253 extending through lower slot 258a. Thus, pins 255, 253 extend through respective slots 256, 258 of grasper handle link 232, openings 248, 250 of grasper handle 230, openings 248a, 250a of grasper handle 230a and slots 256a, 258a of grasper handle link 232a. The grasper links 232, 232a are preferably shaped with a curve as shown so the upper portion containing opening 233, 233a and enlarged region 239, 239a is at an angle to the lower region containing slots 256, 258, 256a, 258a.

As shown in FIG. 21, a proximal collar or barrel 260 and distal collar or barrel 262 are contained within handle assembly 202. The grasper handle links 232, 232a are engageable with proximal collar 260 and the scissor handle links 236, 236a are engageable with distal collar 262 to effect jaw movement as discussed below. The enlarged diameter regions 239, 239a, of grasper handle links 232, 232a, respectively, engage collar 260 (see e.g., FIGS. 18A and 19). The grasper links 232, 232a operatively connect grasper handles 230, 230a with the proximal collar 260 to effect slidable movement of the proximal collar 260 and grasper tube 370 described below for effecting jaw movement in the grasping action. Pivot pin 267 extends through openings 233, 233a at the upper portion of grasper handle links 232, 232a. Grasper links 232, 232a pivot about pin 267 when the grasper handles 230, 230a are moved in a proximal (counterclockwise) direction and a distal (clockwise) direction.

Extending from extension 240 of grasper handle 230 is a hook 252, extending upwardly in the orientation shown, and configured to engage the grasper latch/hook 226 of selector 214. Similarly, extending upwardly from extension 240a of grasper handle 230a is a hook 252a configured to engage grasper latch/hook 226a of selector 214a. When hooks 252, 252a are in locking (blocking) engagement with hooks 226, 226a of selectors 214, 214a, respectively, as shown in FIGS. 18B and 25, the grasper handles 230, 230a are blocked from movement and therefore cannot be actuated to move the jaws 402, 404 in a grasping function. That is, the abutting surfaces of the hooks prevent the grasper handles 230a, 230b from moving proximally to open the jaws 402, 404 in a grasping action. When hooks 252, 252a are not in locking (blocking) engagement with hooks 226, 226a as shown for example in FIGS. 18A and 22, the grasper handles 230, 230a can be actuated (moved) to an open position to open the jaws 402, 404 in a grasping action, i.e., moved in the direction of the arrow of FIG. 23.

Turning now to the scissor handles, scissor (cutter) handle 234 has an upper opening 260, a lower opening 262 and a slot 264. Similarly, scissor (cutter) handle 234a has an upper opening 260a, a lower opening 262a and a slot 264a. Scissor (cutter) handle link 236 has an upper opening 270 and a lower slot 272; scissor (cutter) handle link 236a has an upper opening 270a and a lower slot 272a. The scissor links 270, 270a are preferably shaped at an angle as shown so the upper portion containing openings 270, 270a is at an angle to the lower portion containing slots 272, 272a. The upper portions of scissor handle links 270, 270a engage distal collar 262 to effect slidable movement of the distal collar 262 and cutter tube 372 as the distal collar 262 is moved axially as described in detail below. Pin 274 (FIG. 19) extends through lower slot 272 of scissor handle link 236, lower opening 262 of scissor handle 234, lower opening 262a of scissor handle 234a, and lower slot 272a of scissor handle link 270a. Pivot pin 277 extends through upper opening 270 of scissor handle link 236, upper opening 260 of scissor handle 234, upper opening 260a of scissor handle 234a and upper opening 270a of scissor handle link 236a. Pin 279 extends through slot 264 of scissor handle 234 and slot 264a of scissor handle 234a. Scissor handles 234, 234 pivot in distal (clockwise) and proximal (counterclockwise) direction about pivot pin 277 with slots 264, 264a limiting the extent of travel due to engagement with pin 279.

Scissor handle 234 includes a hook 265 at an upper region engageable with distal hook/latch 224 of selector 214 and scissor handle 234a includes a hook 265a at an upper region engageable with distal hook/latch 224a of selector 214a. When hooks 265, 265a of scissor handles 234, 234a are in locking (blocking) engagement with distal hooks 224, 224a of selector 214, 214a, respectively, the scissor handles 234, 234a cannot be moved and therefore the jaws 402, 404 cannot be moved in the cutting action. This locking of the scissor handles 234, 234a is shown for example in FIGS. 18A and 22. That is, in this locking position, the scissor handles 234, 234a cannot be moved in a distal direction since they are stopped by engagement with hooks 224, 224a of selectors 214, 214a. When the hooks 265, 265a and 224, 224a are not in locking (blocking) engagement as shown for example in FIGS. 18B and 25, there is a gap between the hooks, and the scissor handles 234, 234a can be actuated (moved) to effect jaw movement in a cutting action. It can be appreciated by comparing FIGS. 22 and 25 that when the selectors 214, 214a have lockingly engaged the scissor handles 234, 234a they have freed the grasper handle 230 (and 230a) so the grasper handle 230 (and 230a) can be actuated. Conversely, when the selectors 214, 214a have lockingly engaged the grasper handles 230, 230a, they have freed the scissor handle 234 (and 234a) so the scissor handle 234 (and 234a) can be actuated. Thus, only either the scissor handles or grasper handles can be operated at a time.

There are two positions of the scissor (cutting) handle 234 (the scissor handles 234, 234a are collectively referred to as the scissor handle 234). The scissor handle 234 has a closed position as shown in FIGS. 22-25 and an open position as shown in FIG. 26. In the closed position, the scissor handle 234 is closer to stationary handle 238. In the open position, the scissor handle 234 is pivoted in a distal direction away from stationary handle 238 to open the jaws 402, 404 in a cutting action, the jaw position shown in FIG. 32. When the scissor handle 234 is returned to its more proximal position of FIG. 25 it pivots about pin 277 to move the jaws 402, 404 to the closed position of FIG. 33. When the jaws 402, 404 are moved in the cutting action, preferably the cutting edges touch approximately in the middle portion of the jaws, and in the final position, the tips overlap such that the tip of the top jaw 402 is lower than a tip of the bottom jaw 404. Preferably the force pressing the cutting edge together is the same along a length. Note the range of motion of the scissor handle 230 is limited by the dimension of the lower slot 264 which is engaged by pin 279 as can be appreciated by comparing FIGS. 25 and 26.

There are three positions of the grasper handle 230 (the grasper handles 230, 230a are collectively referred to as the grasper handle 230). The grasper handle 230 has a closed position as shown in FIGS. 18A, 19, 20, and 22 where the jaws 402, 404 are in a closed position of FIG. 29 and an open position wherein the grasper handle 230 is pivoted in a direction away from the stationary handle 238 as shown in FIG. 23 to move the jaws 402, 404 to the open position of FIG. 28. To close the jaws 402, 404 to grasp tissue between the jaws 402, 404, the grasper handle 230 is returned to the position of FIG. 22. Note the jaws 402, 404 have a different radius when they are closed in a grasping action, and preferably only the tips of the jaws touch. Note that in the open and closed positions of the grasper handle 230, the selector 214 cannot be moved to its cutting position because the lower surface of hook 226 (and hook 226a) abuts the upper surface of hook 252 (and hook 252a). Thus, downward movement of the selector 214 to the cutting position is blocked. The grasper handle 230 has a third position which enables the selector 214 to move to the cutting position to enable movement of the jaws 402, 404 in a cutting direction. Note the movement of the grasper handle 230 is limited by the dimension of slot 244 which receives pin 246. This third position of the grasper handle 230 advantageously applies tension to the jaws 402, 404 to enhance the cutting action when the scissor handle 234 is actuated. That is, the jaws bend (are flexible) and the gap between the jaws becomes slightly smaller, causing a preload of the jaws. Due to the different radiuses of the jaws, compensated for by providing a maximum overlap at the tip, during cutting, compression of jaws is reduced resulting in a constant compression of the two jaws 402, 404 so cutting will perform consistently.

In this third “preload” position of the grasper handle 230, the grasper handle 230 is moved distally past its closed position (closer to the stationary handle 238) as shown in FIG. 24. This creates a gap 280 between the selector 214 and the grasping handle 230 to allow for downward movement of the selector 214 to the cutting function position of FIG. 25. Movement of the grasper handle 230 to the third position of FIG. 24 also advances the grasper tube 370 further distally applying a tension to the jaws 402, 404 as yoke 136′, preferably identical to yoke 136 described above, attached, e.g., welded, to grasper tube 370 is moved further forward. In this jaw tensioned position (see FIG. 30), with the yoke 136′ (FIG. 29), providing the extra load to the jaws, the jaws 402, 404 are bent slightly in a longitudinal direction. Thus, such movement applies a compression force to the jaws 402, 404 which enhances cutting when moved in a scissor action.

As noted above, the selector 214 is movable between an upper grasping position and a lower cutting position. In the upper grasping position such as shown in FIG. 24, the round shape ends of springs 222, 222a engage retention slots or notches 315, 315a in back plate 312. Such engagement is illustrated in the close up view of FIG. 27A wherein the selector 214 is in the grasping function position. As can be appreciated, in this position, the scissor handle 234 is locked in position, i.e., blocked from movement, while the grasper handle 230 is free to move. However, the selector 214 cannot be moved downwardly to its lower position due to the abutment of hooks 226 and 252, i.e., the lower surface of hook 226 (and 226a) of the grasper handle abuts the upper surface of hook 252 (and 252a) of selector 214 (and 214a). When the grasper handle 230 is moved to the distal or tensioning position, there is now space for the selector 214 to move into gap 280. The user can then apply a downward force to selector 214, overcoming the bias of the springs 222, 222a. The selector 214 moves downwardly until the springs 222, 222a slide below the back plate 312, where they then snap into position in abutment with the lower surface 313 of the plate 312. This position is shown in FIG. 27B. In this lower position of the selector 214, the grasper handle 230 cannot be moved due to the engagement of the hooks 252, 252a, 226, 226a. However, in this lower position, the scissor handle 234 is free to move due to the gap 282 now created between hook 265 (and 265a) of scissor handle 234 (and 234a) and hook 224 (and 224a) of selector 214 (and selector 214a). If it is desired to move the selector 214 back to the grasping function, the user applies a sufficient upward force to the button (cover) 220 to force the selector 214 upwardly, overcoming the force of retention springs 222 and bypassing hooks 252 (and 252a) of grasper handle 230. The selector 214 is moved upwardly until the round shape ends of springs 222, 222a engage notches 315, 315a in back plate 312 as shown in FIG. 27A. Note that the springs 222, 222a hold the selector 214 in a stable or fixed position and provide a snap in feel as they engage either notches 315, 315a in the grasping position or the lower edge of plate 312 in the cutting position, thus providing in addition to a retention force, a tactile feel to the user so the user knows the selector is set in one of its positions.

The selector 214 cannot be moved between its cutting and grasping positions unless both jaws are in the closed position. That is, if the scissor handle 234 is in the open position, as shown in FIG. 26, the hook 224 (and 224a) of selector 214 will come into abutment with hook 265 (and 265a) of scissor handle 234 and therefore cannot be moved upwardly. If the grasper handle 230 is in the open position, as in FIG. 23, the selector 214 cannot be moved downwardly because the upper surface of hooks 252, 252a still abuts the lower surface of selector 214, 214a.

Turning back to FIG. 21, the remaining components of the handle assembly 202 and how they are assembled/connected will now be described. The handle assembly 202 further includes a pair of left and right side cover plates 302, preferably identical as shown. Front end plate 304 has an opening 306 for passage of the grasper and cuter tubes 370, 372 and opposing side tabs 308 each of which fit into a distal transverse slot 310 of one of the side plates 302 (see also FIG. 20). The back or distal end plate 312 has a slot 314 opening to an upper surface and side tabs 317 on opposing sides, each engageable with a proximal transverse slot 318 of a side plate 302. A pair of openings or notches 315, 315a receive respective retaining springs 222, 222a of selectors 214, 214a to retain the selectors 214, 214a in the grasping function position as described above. Intermediate plate 320, positioned distal of back plate 312, has a slot 322 opening into a lower surface and opposing side tabs 324, each engageable with a transverse slot 316, positioned distal of transverse slot 318, formed in side plates 302. Connecting plate 328 (see also FIG. 34) has a pair of side tabs 334 engageable with slots 227a of selector 214a, and a pair of side tabs 336 on the opposing side engageable with slots 227 of selector 214 to retain the two selectors 214, 214a together forming a single selector 214 movable as a single unit as discussed above.

With continued reference to FIG. 21, proximal barrel or collar 260 has a distal flange 350 and a proximal flange 352, forming a reduced diameter portion 354 in between. Opening 356 is dimensioned to receive and retain grasper tube 370 so that movement of the barrel 260 moves the grasper tube 370. Enlarged diameter regions 239, 239a of grasper handle links 232, 232a are positioned within reduced portion 354 of barrel 260 so that movement of the grasper handle links 232, 232a moves barrel 260 which in turn moves grasper tube 370 which effects movement of the jaws 402, 404 in a grasping direction. Distal barrel or collar 262 has a distal flange 360 and a proximal flange 362, forming a reduced diameter region 366 in between. Opening 368 in barrel 262 is dimensioned to receive and retain cutter tube 372. The upper portion of scissor handle links 236, 236a, are positioned within reduced portion 366 of barrel 262 so that movement of the scissor handle links 236, 236a moves barrel (distal end tube) 262 which in turn moves cutter tube 372 which effects movement of the jaws 402, 404 in a cutting direction. Grasper tube 370 is connected within barrel 260 and is operably connected to the jaws 402, 404 in the same manner as grasper tube 140 described above, e.g., a yoke like yoke 136 with openings 131a, 131b to receive the jaw pins. Cutter tube 372 is connected within distal barrel 262 and is operably connected to the jaws 402, 404 in the same manner as cutter tube 150 described above, e.g., extensions on the tube with openings to receive the jaws pins as in elements 150, 152a, 152b and 153a, 153b as described above. Therefore, for brevity the connection to the jaw pins will not be repeated since the connection described above with respect to the embodiments of FIGS. 1-16 is fully applicable to the embodiment of FIGS. 17-34.

The use of the instrument 200 will now be described. The surgical instrument 200 is preferably packaged in the position of FIGS. 18A and 19. In this position, the grasper handle 230 is ready for actuation, e.g., movement in the grasping function to an open position to open the jaws 402, 404 to receive tissue and back to a closed position to close the jaws 402, 404 to grasp tissue between the jaws 402, 404. In this position, the selector retention springs 222, 222a are engaged in the retention notches 315, 315a of back plate 312 (see FIG. 27A). Note, in this position, the selector 214, 214a cannot be pivoted downwardly to its cutting position because, as shown in FIG. 22, the upper surface of the hook 252 (and 252a) of grasper handle 230 (and 230a) blocks downward movement of the selector 214 (and 241a). That is, the lower surface of proximal hooks 226 and 226a of selector 214 and 214a will abut the upper surface of the hook 252 and 252a if downward movement is attempted. Also note in this position the scissor handle 234 is blocked from movement because of the locking (or blocking) engagement of hook 265 (and 265a) of scissor handle 234 (and 234a) with distal hook 224 (and 224a) of selector 214 (and 214a). Further note in this position the pin 246 is in a proximal region of slot 244 (and 244a) of grasper handle 230 (and 230a), but spaced from the proximalmost edge (see FIG. 22).

If the user desires to effect grasping of tissue or other structure, the grasping handle 230 is moved in a proximal or counterclockwise direction as shown in FIG. 23, pivoting about pin 267 (which also engages the grasper handle links 232, 232a), causing the enlarged diameter region 239 and 239a of grasper handle links 232 and 232a, engaged in the reduced diameter region 354 of barrel 260, to apply a force to proximal flange 352 to move the barrel 260 and attached grasper tube 370 axially in a proximal direction to the position of FIG. 23. In this position, pin 246 is now located in the distalmost region of slot 244 (and 244a) of grasper handles 230 (and 230a), thereby limiting the extent of travel of the grasper handle 230. When the grasper tube 370 is moved in the proximal direction, the jaws 402, 404 move from the closed position of FIG. 29 to the open position of FIG. 28. Note in this position the selector 214 (and 214a) remains blocked from movement by hooks 252 (and 252a). To close the jaws 402, 404 in a grasping function to the position of FIG. 29, the grasper handle 230 is then returned to its initial position of FIG. 22, with the enlarged diameter region 239 and 239a of grasper handle link 232 and 232a applying a force to the distal flange 350 of barrel 260 to move the barrel 260 axially in a distal direction, thereby moving the connected grasper tube 370 distally to its original position. The user can continue to move (open and close) the jaws 402, 404 in the grasping direction by repeated actuation of grasper handle 230 if desired.

If the user desires to transition to the scissor (cutting) function, the grasper handle 230 is moved to its more distal position of FIG. 24, referred to above for convenience as the third position. As it moves to this distal position, the enlarged regions 239, 239a of the grasper handle links 232, 232a contact the distal flange 350 of barrel 260 to apply a forward (distal) force to the barrel 260 to move the grasping tube 370 and attached yoke 136′ so it extends further toward the jaws 402, 404, as shown in FIG. 33, causing a slight bend and compression in the jaws 402, 404, thereby tensioning the jaws 402, 404 as described above.

As can be appreciated by comparing FIGS. 23 and 24, in this distal jaw tensioning position of FIG. 24, the pin 246 is in the proximalmost position within slot 244 (and 244a). Thus, this pin/slot engagement limits the extent of distal movement of grasper handle 230. Also, as shown, this further movement of the grasper handle 230 to the distal position moves the upper blocking surface of the hook 252 (and 252a) of the grasper handle 230 slightly away from the selector 214 (downwardly as viewed in the orientation of FIG. 24). This provides a gap 280 so the selector button or tab 220 can be moved downwardly by the user, overriding the force of the retention springs 222, 222a. The selector 214 is moved downwardly in the direction of the arrow of FIG. 25 until retention springs 222, 222a snap under the rear plate 312 (see FIG. 27B). This lower cutting position of the selector 214 is shown in FIGS. 18B and 25. Note that the movement of the springs 222, 222a into the lower position provides a tactile feel to the user as noted above. In this lower position, the hook 226 (and 226a) of selector 214 (and 214a) are in locking (blocking) engagement with hooks 252 (and 252a) so that the grasper handle 230 cannot be moved to an open position. In this lower position, there is a gap 282 between hook 265 (and 265a) of scissor handle 230 (and 230a) and hook 224 (and 224a) of selector 214 (and 214a). This enables the scissor handle 234 to be moved in a cutting function. That is, the user can now open the jaws 402, 404 in a scissor action (FIG. 32) by moving scissor handle 234 in a distal direction shown in FIG. 26, thereby causing the upper portion of scissor handle links 236, 236a to engage proximal flange 362 of barrel 262 to move cutter tube 372 proximally to move the jaws 402, 404 from the closed position of FIG. 31 to the open position of FIG. 32.

To close the jaws 402, 404 to sever tissue or other structure in a cutting action, scissor handle 234 is moved back to the position of FIG. 25, thereby causing handle links 236, 236a to engage the distal flange 360 of barrel 262, forcing the connected cutter tube 372 in the distal direction to return the jaws 402, 404 to the position of FIG. 33. The user can continue, if desired, to open and close the jaws in the cutting action by repeated actuation of scissor handle 234 if desired. Note that in the closed position of FIG. 25, pin 279 is in a distalmost region of the slot 264 (and 264a) of the handle 234 (and 234a) and in the open position the pin 279 is in the proximalmost region of slot 264 (and 264a). Thus, the travel of scissor handle 234 is limited by this pin/slot arrangement.

If the user wants to move to the grasping function, with the scissor handle 234 in the closed position, the user applies an upward force to the button 220, overcoming the biasing force of springs 222, 222a. The user pivots the selector 214 upwardly until springs 222, 222a engage the retention notches 315, 315a in the plate 312. This helps retain the selector 214 in the grasping function as noted above. The user can then actuate grasper handle 230 in a grasping function.

In one embodiment, the handle components are formed by cutting, e.g., laser cutting, one or more metal sheets. In the embodiment shown in FIG. 34 the following components are formed from a single sheet of metal: grasper handles 230, 230a, grasper links 232, 232a, scissor handles 234, 234a, scissor handle links 236, 236a, selectors 214, 214a with springs 222, 222a, front, back and intermediate plates 304, 320 and 312, selector connector plate 328 and the two side plates 302. This manufacturing method provides a faster and less expensive manufacturing process. It also provides for a lighter weight handle assembly. It should be appreciated that although in one embodiment, these listed components are formed from a single sheet, it is also contemplated they are formed from two or more sheets. Additionally, fewer or more of the handle components can be manufactured in this method, i.e., from one or more metal sheets. Although laser cutting is a preferred method, other methods for forming these shaped components are also contemplated.

It should be appreciated that the instrument can alternatively be provided with different jaws to perform other functions beside grasping and cutting. The switching mechanism would enable switching between the two different functions.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

What is claimed is:

1. A surgical instrument for cutting and grasping tissue comprising:
a handle assembly disposed at a proximal portion of the instrument;
an elongated member extending from the handle assembly;
a first jaw positioned adjacent a distal portion of the elongated member;
a second jaw positioned adjacent the distal portion of the elongated member, at least one of the first or second jaws movable with respect to the other jaw;
a first movable member operably associated with at least one of the first and second jaws, the first movable member movable between first and second positions to effect jaw movement in a grasping action;
a second movable member operably associated with at least one of the first and second jaws, the second movable member movable between first and second positions to effect jaw movement in a cutting action; and
a selector engageable with the handle assembly and movable from a first position to a second position, wherein in the first position of the selector, the selector is in blocking engagement with the handle assembly to prevent jaw movement in the cutting action and in the second position of the selector, the selector is in blocking engagement with the handle assembly to prevent jaw movement in the grasping action.

2. The instrument of claim 1, wherein movement of at least one of the first and second jaws in the grasping action is about a first axis and movement of at least one of the first and second jaws in the cutting action is about a second different axis.

3. The instrument of claim 2, wherein the first axis and the second axis intersect and are substantially perpendicular.

4. The instrument of claim 1, wherein the selector is not movable from the first position to the second position if the jaws are in an open position.

5. The instrument of claim 1, wherein the handle assembly includes a first handle movable in a first direction to effect jaw movement to an open position and movable in a second direction to effect jaw movement to a closed position in the grasping action.

6. The instrument of claim 5, wherein the first handle is movable further in the second direction to apply increased tension to the first and second jaws prior to the cutting action.

7. The instrument of claim 6, wherein movement of the first handle further in the second direction causes compression of the first and second jaws.

8. The instrument of claim 6, wherein the selector cannot be moved to the second position unless the first handle is moved to a position to apply increased tension to the first and second jaws.

9. The instrument of claim 1, wherein the handle assembly includes an engagement surface engageable with the selector, the engagement surface preventing movement of the selector.

10. The instrument of claim 1, wherein the selector includes a spring, the spring retaining the selector in the first position and in the second position.

11. The instrument of claim 1, wherein the selector is pivotable between the first and second positions of the selector.

12. A surgical instrument for cutting and grasping tissue comprising:
an elongated member having a distal portion;
a first jaw positioned adjacent the distal portion of the elongated member;
a second jaw positioned adjacent the distal portion of the elongated member and movable with respect to the first jaw;
a first movable member operably associated with at least the second jaw, the first movable member having a first position wherein the second jaw is spaced from the first jaw, a second position wherein the second jaw is moved toward to the first jaw in a grasping action, and a third position wherein the first and second jaws are brought under increased tension and bend;
a second movable member operably associated with at least the second jaw, the second movable member movable from a first position to a second position to move the second jaw toward the first jaw in a cutting action; and
a selector positioned at a proximal portion of the instrument to enable movement of either the first movable member or the second movable member.

13. The instrument of claim 12, wherein the second movable member is movable only after the first movable member is in the third position.

14. The instrument of claim 12, wherein the selector is movable to enable movement of the second movable member in a cutting action only after the first movable member has been moved to the third position.

15. The instrument of claim 12, wherein the first movable member is a first handle, the first handle operatively connected to a first tube movable axially to effect movement of at least the second jaw in the grasping action and the second movable member is a second handle, the second handle operatively connected to a second tube movable axially to effect movement of at least the second jaw in the cutting action.

16. The instrument of claim 14, wherein the first movable member is a first axially slidable tubular member and the second movable member is a second axially slidable tubular member.

17. A surgical instrument actuation mechanism formed from one or more metal sheets, the one or more metal sheets formed to have a first handle operable to effect a jaw grasping action, a second handle operable to effect a jaw cutting action, a first link operably connected to the first handle, a second link operably connected to the second handle, and a selector operably connected to the first handle and the second handle to select handle movement in the grasping action or the cutting action.

18. The surgical instrument of claim 17, wherein the first handle formed from the one or more metal sheets includes first and second handles each having a finger loop and the second handle formed from the one or more metal sheets includes first and second handles each having a finger loop.

19. The surgical instrument of claim 17, further comprising first and second side plates formed from the one or more metal sheets.

20. The surgical instrument of claim 19, wherein the selector includes first and second connectors, the connector plate joining the first and second connectors.
